SEQUENCE LISTING (60 sequences)

SEQ ID NO: 1 - protein, 25 aa, Artificial Sequence
  Description of Artificial Sequence Synthetic peptide
DTHFPICIFCCGCCHRSKCGMCCKT

SEQ ID NO: 2 - protein, 21 aa, Artificial Sequence
  Description of Artificial Sequence Synthetic peptide
PICIFCCGCCHRSKCGMCCKT

SEQ ID NO: 3 - protein, 19 aa, Artificial Sequence
  Description of Artificial Sequence Synthetic peptide
PICIFCCGCCHRSKCGMCC

SEQ ID NO: 4 - protein, 20 aa, Artificial Sequence
  Description of Artificial Sequence Synthetic peptide
ICIFCCGCCHRSKCGMCCKT

SEQ ID NO: 5 - protein, 17 aa, Artificial Sequence
  Description of Artificial Sequence Synthetic peptide
CIFCCGCCHRSKCGMCC

SEQ ID NO: 6 - protein, 25 aa, Artificial Sequence
  Description of Artificial Sequence Synthetic peptide
  MOD_RES (1)..(2): Any amino acid
  MOD_RES (4)..(4): Any amino acid
  MOD_RES (6)..(6): Any amino acid
  MOD_RES (8)..(9): Any amino acid
  MOD_RES (24)..(25): Any amino acid
XXHXPXCXXCCGCCHRSKCGMCCXX

SEQ ID NO: 7 - protein, 21 aa, Artificial Sequence
  Description of Artificial Sequence Synthetic peptide
  MOD_RES (2)..(2): Any amino acid
  MOD_RES (4)..(5): Any amino acid
  MOD_RES (21)..(21): Any amino acid
PXCXXCCGCCHRSKCGMCCKX

SEQ ID NO: 8 - protein, 19 aa, Artificial Sequence
  Description of Artificial Sequence Synthetic peptide
  MOD_RES (2)..(2): Any amino acid
  MOD_RES (4)..(5): Any amino acid
PXCXXCCGCCHRSKCGMCC

SEQ ID NO: 9 - protein, 20 aa, Artificial Sequence
  Description of Artificial Sequence Synthetic peptide
  MOD_RES (1)..(1): Any amino acid
  MOD_RES (3)..(4): Any amino acid
  MOD_RES (12)..(13): Any amino acid
  MOD_RES (16)..(16): Any amino acid
  MOD_RES (20)..(20): Any amino acid
XCXXCCGCCHRXXCGXCCKX

SEQ ID NO: 10 - protein, 17 aa, Artificial Sequence
  Description of Artificial Sequence Synthetic peptide
  MOD_RES (2)..(3): Any amino acid
  MOD_RES (11)..(12): Any amino acid
  MOD_RES (15)..(15): Any amino acid
CXXCCGCCHRXXCGXCC

SEQ ID NO: 11 - protein, 6 aa, Artificial Sequence
  Description of Artificial Sequence Synthetic peptide
HFPICI

SEQ ID NO: 12 - protein, 7 aa, Artificial Sequence
  Description of Artificial Sequence Synthetic peptide
HFPICIF

SEQ ID NO: 13 - protein, 17 aa, Artificial Sequence
  Description of Artificial Sequence Synthetic peptide
DTHFPICIDTHFPICIF

SEQ ID NO: 14 - protein, 10 aa, Artificial Sequence
  Description of Artificial Sequence Synthetic peptide
DTHFPIAIFC

SEQ ID NO: 15 - protein, 9 aa, Artificial Sequence
  Description of Artificial Sequence Synthetic peptide
DTHAPICIF

SEQ ID NO: 16 - protein, 9 aa, Artificial Sequence
  Description of Artificial Sequence Synthetic peptide
DTHFPICIF

SEQ ID NO: 17 - protein, 10 aa, Artificial Sequence
  Description of Artificial Sequence Synthetic peptide
CDTHFPICIF

SEQ ID NO: 18 - protein, 9 aa, Artificial Sequence
  Description of Artificial Sequence Synthetic peptide
  MOD_RES (1)..(9): D-amino acid
FICIPFHTD

SEQ ID NO: 19 - protein, 7 aa, Artificial Sequence
  Description of Artificial Sequence Synthetic peptide
  MOD_RES (1)..(7): D-amino acid
FICIPFH

SEQ ID NO: 20 - protein, 9 aa, Artificial Sequence
  Description of Artificial Sequence Synthetic peptide
  MOD_RES (1)..(9): D-amino acid
FICIPFHTD

SEQ ID NO: 21 - protein, 9 aa, Artificial Sequence
  Description of Artificial Sequence Synthetic peptide
  MOD_RES (1)..(9): D-amino acid
FICIPFHTD

SEQ ID NO: 22 - protein, 9 aa, Artificial Sequence
  Description of Artificial Sequence Synthetic peptide
  MOD_RES (1)..(9): D-amino acid
FICIPFHTD

SEQ ID NO: 23 - protein, 9 aa, Artificial Sequence
  Description of Artificial Sequence Synthetic peptide
  MOD_RES (1)..(9): D-amino acid
FICIPFHTD

SEQ ID NO: 24 - protein, 9 aa, Artificial Sequence
  Description of Artificial Sequence Synthetic peptide
  MOD_RES (1)..(9): D-amino acid
FICIPFHTD

SEQ ID NO: 25 - protein, 12 aa, Artificial Sequence
  Description of Artificial Sequence Synthetic peptide
  MOD_RES (1)..(1): May or may not be present
  MOD_RES (2)..(2): Iminodiacetic acid
  MOD_RES (5)..(5): 3,3-diphenyl-L-alanine
  MOD_RES (6)..(6): Beta-homoproline, L-nipecotic acid, D-nipecotic acid, isonipecotic acid, or beta-alanine
  MOD_RES (11)..(11): Aminohexanoic acid
  MOD_RES (12)..(12): Iminodiacetic acid(NH-PAL)
GXTHAXRCRWXX

SEQ ID NO: 26 - protein, 12 aa, Artificial Sequence
  Description of Artificial Sequence Synthetic peptide
  MOD_RES (1)..(1): Iminodiacetic acid(NH-PAL)
  MOD_RES (2)..(2): Aminohexanoic acid
  MOD_RES (7)..(7): Beta-homoproline, L-nipecotic acid, D-nipecotic acid, isonipecotic acid, or beta-alanine
  MOD_RES (8)..(8): 3,3-diphenyl-L-alanine
  MOD_RES (11)..(11): Iminodiacetic acid
  MOD_RES (12)..(12): May or may not be present
XXWRCRXAHTXG

SEQ ID NO: 27 - protein, 11 aa, Artificial Sequence
  Description of Artificial Sequence Synthetic peptide
  MOD_RES (1)..(1): Iminodiacetic acid
  MOD_RES (4)..(4): 3,3-diphenyl-L-alanine
  MOD_RES (5)..(5): Beta-homoproline
  MOD_RES (10)..(10): Aminohexanoic acid
  MOD_RES (11)..(11): Iminodiacetic acid(NH-PAL)
XTHAPRCRWXX

SEQ ID NO: 28 - protein, 11 aa, Artificial Sequence
  Description of Artificial Sequence Synthetic peptide
  MOD_RES (1)..(1): Iminodiacetic acid
  MOD_RES (4)..(4): 3,3-diphenyl-L-alanine
  MOD_RES (5)..(5): Beta-homoproline
  MOD_RES (10)..(10): Aminohexanoic acid
  MOD_RES (11)..(11): Iminodiacetic acid(NH-PAL)
XTHAPRCRWXX

SEQ ID NO: 29 - protein, 11 aa, Artificial Sequence
  Description of Artificial Sequence Synthetic peptide
  MOD_RES (1)..(1): Iminodiacetic acid
  MOD_RES (4)..(4): 3,3-diphenyl-L-alanine
  MOD_RES (5)..(5): Beta-homoproline
  MOD_RES (10)..(10): Aminohexanoic acid
  MOD_RES (11)..(11): Iminodiacetic acid(NH-PAL)
XTHAPRCRWXX

SEQ ID NO: 30 - protein, 11 aa, Artificial Sequence
  Description of Artificial Sequence Synthetic peptide
  MOD_RES (1)..(1): Iminodiacetic acid
  MOD_RES (4)..(4): 3,3-diphenyl-L-alanine
  MOD_RES (5)..(5): L-nipecotic acid
  MOD_RES (10)..(10): Aminohexanoic acid
  MOD_RES (11)..(11): Iminodiacetic acid(NH-PAL)
XTHAXRCRWXX

SEQ ID NO: 31 - protein, 11 aa, Artificial Sequence
  Description of Artificial Sequence Synthetic peptide
  MOD_RES (1)..(1): Iminodiacetic acid
  MOD_RES (4)..(4): 3,3-diphenyl-L-alanine
  MOD_RES (5)..(5): L-nipecotic acid
  MOD_RES (10)..(10): Aminohexanoic acid
  MOD_RES (11)..(11): Iminodiacetic acid(NH-PAL)
XTHAXRCRWXX

SEQ ID NO: 32 - protein, 11 aa, Artificial Sequence
  Description of Artificial Sequence Synthetic peptide
  MOD_RES (1)..(1): Iminodiacetic acid
  MOD_RES (4)..(4): 3,3-diphenyl-L-alanine
  MOD_RES (5)..(5): D-nipecotic acid
  MOD_RES (10)..(10): Aminohexanoic acid
  MOD_RES (11)..(11): Iminodiacetic acid(NH-PAL)
XTHAXRCRWXX

SEQ ID NO: 33 - protein, 11 aa, Artificial Sequence
  Description of Artificial Sequence Synthetic peptide
  MOD_RES (1)..(1): Iminodiacetic acid
  MOD_RES (4)..(4): 3,3-diphenyl-L-alanine
  MOD_RES (5)..(5): Isonipecotic acid
  MOD_RES (10)..(10): Aminohexanoic acid
  MOD_RES (11)..(11): Iminodiacetic acid(NH-PAL)
XTHAXRCRWXX

SEQ ID NO: 34 - protein, 12 aa, Artificial Sequence
  Description of Artificial Sequence Synthetic peptide
  MOD_RES (2)..(2): Iminodiacetic acid
  MOD_RES (5)..(5): 3,3-diphenyl-L-alanine
  MOD_RES (6)..(6): Beta-homoproline
  MOD_RES (11)..(11): Aminohexanoic acid
  MOD_RES (12)..(12): Iminodiacetic acid(NH-PAL)
GXTHAPRCRWXX

SEQ ID NO: 35 - protein, 11 aa, Artificial Sequence
  Description of Artificial Sequence Synthetic peptide
  MOD_RES (1)..(1): Iminodiacetic acid
  MOD_RES (4)..(4): 3,3-diphenyl-L-alanine
  MOD_RES (5)..(5): Beta-alanine
  MOD_RES (10)..(10): Aminohexanoic acid
  MOD_RES (11)..(11): Iminodiacetic acid(NH-PAL)
XTHAARCRWXX

SEQ ID NO: 36 - protein, 27 aa, Artificial Sequence
  Description of Artificial Sequence Synthetic peptide
GYIPEAPRDGQAYVRKDGEWVLLSTFL

SEQ ID NO: 37 - protein, 30 aa, Artificial Sequence
  Description of Artificial Sequence Synthetic polypeptide
  MOD_RES (3)..(3): Hydroxyproline
  MOD_RES (6)..(6): Hydroxyproline
  MOD_RES (9)..(9): Hydroxyproline
  MOD_RES (12)..(12): Hydroxyproline
  MOD_RES (15)..(15): Hydroxyproline
  MOD_RES (18)..(18): Hydroxyproline
  MOD_RES (21)..(21): Hydroxyproline
  MOD_RES (24)..(24): Hydroxyproline
  MOD_RES (27)..(27): Hydroxyproline
  MOD_RES (30)..(30): Hydroxyproline
GPPGPPGPPGPPGPPGPPGPPGPPGPPGPP

SEQ ID NO: 38 - protein, 9 aa, Artificial Sequence
  Description of Artificial Sequence Synthetic peptide
  MOD_RES (7)..(7): L-homocysteine
DTHFPICIF

SEQ ID NO: 39 - protein, 9 aa, Artificial Sequence
  Description of Artificial Sequence Synthetic peptide
  MOD_RES (7)..(7): L-penicillamine
DTHFPIXIF

SEQ ID NO: 40 - protein, 9 aa, Artificial Sequence
  Description of Artificial Sequence Synthetic peptide
  MOD_RES (2)..(2): L-tert-leucine
  MOD_RES (4)..(4): L-phenylglycine
  MOD_RES (5)..(5): Octahydroindole-2-carboxylic acid
  MOD_RES (6)..(6): L-alpha-cyclohexylglycine
  MOD_RES (8)..(8): L-alpha-cyclohexylglycine
DLHGXGCGF

SEQ ID NO: 41 - protein, 9 aa, Artificial Sequence
  Description of Artificial Sequence Synthetic peptide
  MOD_RES (2)..(2): L-tert-leucine
  MOD_RES (5)..(5): Octahydroindole-2-carboxylic acid
  MOD_RES (6)..(6): L-alpha-cyclohexylglycine
  MOD_RES (8)..(8): L-alpha-cyclohexylglycine
DLHPXGCGF

SEQ ID NO: 42 - protein, 9 aa, Artificial Sequence
  Description of Artificial Sequence Synthetic peptide
  MOD_RES (4)..(4): (S)-2-amino-4-phenylbutanoic acid
DTHXPICIF

SEQ ID NO: 43 - protein, 9 aa, Artificial Sequence
  Description of Artificial Sequence Synthetic peptide
  MOD_RES (4)..(4): 3,3-diphenyl-L-alanine
DTHAPICIF

SEQ ID NO: 44 - protein, 9 aa, Artificial Sequence
  Description of Artificial Sequence Synthetic peptide
  MOD_RES (4)..(4): L-biphenylalanine
DTHXPICIF

SEQ ID NO: 45 - protein, 9 aa, Artificial Sequence
  Description of Artificial Sequence Synthetic peptide
  MOD_RES (4)..(4): (1-naphthyl)-L-alanine
DTHAPICIF

SEQ ID NO: 46 - protein, 9 aa, Artificial Sequence
  Description of Artificial Sequence Synthetic peptide
  MOD_RES (4)..(4): (S)-3-amino-4,4-diphenylbutanoic acid
DTHXPICIF

SEQ ID NO: 47 - protein, 9 aa, Artificial Sequence
  Description of Artificial Sequence Synthetic peptide
  MOD_RES (9)..(9): (S)-2-amino-4-phenylbutanoic acid
DTHFPICIX

SEQ ID NO: 48 - protein, 9 aa, Artificial Sequence
  Description of Artificial Sequence Synthetic peptide
  MOD_RES (9)..(9): 3,3-diphenyl-L-alanine
DTHFPICIA

SEQ ID NO: 49 - protein, 9 aa, Artificial Sequence
  Description of Artificial Sequence Synthetic peptide
  MOD_RES (9)..(9): L-biphenylalanine
DTHFPICIX

SEQ ID NO: 50 - protein, 9 aa, Artificial Sequence
  Description of Artificial Sequence Synthetic peptide
  MOD_RES (9)..(9): (1-naphthyl)-L-alanine
DTHFPICIA

SEQ ID NO: 51 - protein, 9 aa, Artificial Sequence
  Description of Artificial Sequence Synthetic peptide
  MOD_RES (9)..(9): (S)-3-amino-4,4-diphenylbutanoic acid
DTHFPICIX

SEQ ID NO: 52 - protein, 9 aa, Artificial Sequence
  Description of Artificial Sequence Synthetic peptide
  MOD_RES (4)..(4): 3,3-diphenyl-L-alanine
  MOD_RES (9)..(9): 3,3-diphenyl-L-alanine
DTHAPICIA

SEQ ID NO: 53 - protein, 7 aa, Artificial Sequence
  Description of Artificial Sequence Synthetic peptide
  MOD_RES (2)..(2): 3,3-diphenyl-L-alanine
DAPICIF

SEQ ID NO: 54 - protein, 7 aa, Artificial Sequence
  Description of Artificial Sequence Synthetic peptide
  MOD_RES (2)..(2): 3,3-diphenyl-L-alanine
  MOD_RES (7)..(7): 3,3-diphenyl-L-alanine
DAPICIA

SEQ ID NO: 55 - protein, 9 aa, Artificial Sequence
  Description of Artificial Sequence Synthetic peptide
  MOD_RES (4)..(4): 3,3-diphenyl-L-alanine
  MOD_RES (9)..(9): 3,3-diphenyl-L-alanine
DTHAPRCRA

SEQ ID NO: 56 - protein, 9 aa, Artificial Sequence
  Description of Artificial Sequence Synthetic peptide
  MOD_RES (4)..(4): 3,3-diphenyl-L-alanine
  MOD_RES (5)..(5): Octahydroindole-2-carboxylic acid
DTHAXICIF

SEQ ID NO: 57 - protein, 9 aa, Artificial Sequence
  Description of Artificial Sequence Synthetic peptide
  MOD_RES (4)..(4): 3,3-diphenyl-L-alanine
  MOD_RES (5)..(5): Octahydroindole-2-carboxylic acid
  MOD_RES (9)..(9): 3,3-diphenyl-L-alanine
DTHAXICIA

SEQ ID NO: 58 - protein, 9 aa, Artificial Sequence
  Description of Artificial Sequence Synthetic peptide
  MOD_RES (4)..(4): 3,3-diphenyl-L-alanine
  MOD_RES (9)..(9): 3,3-diphenyl-L-alanine
DTHAPCCCA

SEQ ID NO: 59 - protein, 12 aa, Artificial Sequence
  Description of Artificial Sequence Synthetic peptide
  MOD_RES (10)..(10): L-beta-homoproline
DTHFPICIFPPK

SEQ ID NO: 60 - protein, 12 aa, Artificial Sequence
  Description of Artificial Sequence Synthetic peptide
  MOD_RES (10)..(10): L-beta-homoproline
DTHFPICIFPPR

(19) United States
(12) Patent Application Publication
(10) Pub. No.: US 2018/0099023 A1
(43) Pub. Date: Apr. 12, 2018

(54) METHODS OF TREATING IRON OVERLOAD

(71) Applicant: La Jolla Pharmaceutical Company, San Diego, CA (US)

(72) Inventors: George Tidmarsh, Portola Valley, CA (US); Lakhmir Chawla, San Diego, CA (US)

(21) Appl. No.: 15/697,325

(22) Filed: Sep. 6, 2017

Related U.S. Application Data
(60) Provisional application No. 62/383,957, filed on Sep. 6, 2016.

(51) Int. Cl. A61K 38/17; A61P 43/00; A61P 7/00; A61K 9/00

(52) U.S. Cl. CPC A61K 38/1709; A61K 9/0019; A61P 7/00; A61P 43/00

(57) ABSTRACT

Provided herein are compositions and methods related to the use of hepcidin and/or hepcidin analogues for the treatment and/or prevention of iron overload in a subject (e.g., a human subject) and/or for reducing serum iron levels in a subject without inducing serum iron rebound.

### RELATED APPLICATIONS

This application claims the benefit of priority to U.S. Provisional Patent Application Ser. No. 62/383,957, filed Sep. 6, 2016, which is herein incorporated by reference in its entirety.

### BACKGROUND

Iron is an essential element required for growth and survival of almost every organism. In mammals, the iron balance is primarily regulated at the level of duodenal absorption of dietary iron. Following absorption, ferric iron is loaded into apo-transferrin in the circulation and transported to the tissues, including erythroid precursors, where it is taken up by transferrin receptor-mediated endocytosis. Reticuloendothelial macrophages play a major role in the recycling of iron from the degradation of hemoglobin of senescent erythrocytes, while hepatocytes contain most of the iron stores of the organism in ferritin polymers. In the case of iron deficiency, the pathophysiological consequences of gene defects identified are well understood because they usually result in loss of function of proteins directly involved in the pathway of iron absorption. The proteins include the iron transporters DMT1 (also called Nramp2 or DCT1), ferroportin (also called IREG1 or MTP1), and copper oxidases coupled to ferroportin, namely ceruloplasmin and haephastin.

Iron overload (also referred to as hemochromatosis) is the exact opposite of iron deficiency, and refers to the over-accumulation of iron in the body. Chronic iron overload can lead to a number of detrimental conditions, including cirrhosis of the liver, diabetes, cardiomyopathy and arthritis. The genetic disorder hereditary hemochromatosis (HHC) is a relatively common autosomal recessive genetic disease that results in the hyperabsorption of dietary iron leading to an iron overload in plasma and organs. The excess iron is stored in the body's tissues and organs, particularly the skin, heart, liver, pancreas, and joints. Because humans cannot increase the excretion of iron, excess iron can overload and eventually damage tissues and organs. Once diagnosed, hemochromatosis is often treated by phlebotomy to rid the body of excess iron and to maintain normal iron stores. Phlebotomy, an invasive and inefficient therapy, remains the sole recommended treatment for hereditary hemochromatosis.

Beta thalassemias (β thalassemias) are a group of inherited blood disorders caused by reduced or absent synthesis of the beta chains of hemoglobin that result in outcomes ranging from severe anemia to clinically asymptomatic individuals. Current treatments include repeated blood transfusions, which can result in transfusional iron overload. In many patients with β thalassemia, multiple blood transfusions, ineffective erythropoiesis, and increased gastrointestinal iron absorption lead to iron overload in the body. Iron overload impairs the immune system, placing patients at greater risk of infection and illness. To counter this iron overload, patients often undergo chelation therapy. Many common chelators used for treating iron overload are associated with toxicity and renal impairment, hepatic impairment and gastrointestinal hemorrhage. Thus, there is a need for new treatments for iron overload and related disorders that are safer and better tolerated.

### SUMMARY

Provided herein are compositions and methods related to the use of hepcidin and/or hepcidin analogues for the treatment and/or prevention of iron overload in a subject (e.g., a human subject) and/or for reducing serum iron levels in a subject. The compositions and methods provided herein are related, in part, to the discovery of serum iron level rebound following hepcidin therapy under some conditions. Following hepcidin administration at higher doses, some patients experience a serum iron level rebound, in which serum iron levels initially drop in response to hepcidin administration, but then paradoxically rise (or rebound) above baseline iron level (i.e., the level of serum iron prior to hepcidin administration). The compositions and methods described herein follow from the observation that this undesirable rebound in serum iron level can be avoided or mitigated by initially administering low doses of hepcidin (e.g., 1-20 mg, preferably 1-10 mg, or even more preferably 1-5 mg), and/or increasing the frequency of administration of lower doses to heighten the effects of hepcidin therapy (rather than administer doses above 40 mg, or even above 30 mg or even above 20 mg). The need for increasing the frequency or dose of hepcidin administration can be identified from measurements of serum or tissue iron levels in the patient (for example, serum iron levels, ferritin levels, transferrin saturation, hemoglobin, or hematocrit) and comparing these measurements to predetermined target levels.

In certain embodiments, the methods provided herein include administering to the subject hepcidin or a hepcidin analogue at an amount sufficient to reduce the serum iron concentration in the subject without inducing a serum iron level rebound following treatment.

In some embodiments, the amount of hepcidin or hepcidin analogue is between about 0.1 mg and about 40 mg. In some embodiments, the amount of hepcidin or hepcidin analogue is between 1 mg and about 30 mg (e.g., between about 5 mg and about 30 mg, between about 10 mg and about 30 mg, between about 20 mg and about 30 mg, between about 1 mg and about 20 mg, between about 5 mg and about 20 mg, between about 10 mg and about 20 mg, between about 1 mg and about 10 mg, between about 5 mg and about 10 mg). In some embodiments, the amount of hepcidin or hepcidin analogue is about 0.1 mg, 0.5 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg or 40 mg.

Provided herein are methods of treating or preventing iron overload in a subject and/or reducing serum iron levels in a subject by administering to the subject a hepcidin or hepcidin analogue at an amount sufficient to reduce the serum iron concentration of the subject, wherein administering the hepcidin or hepcidin analogue comprises administering the hepcidin or hepcidin analogue at an initial dose below the threshold to induce serum iron rebound. In some embodiments, the methods further comprise administering to the subject an additional dose or doses of the hepcidin or hepcidin analogue, e.g., on a periodic basis (e.g., biweekly, weekly, semiweekly, daily), which doses may be the same as the initial dose or higher or lower depending on whether the patient has experienced the desired clinical response. The additional dose or doses may be below the threshold to induce serum iron rebound (e.g., 40 mg or less, preferably 30 mg or less, or even 20 mg or less). The additional dose or doses may be the same as the threshold dose to induce serum iron rebound. The additional dose or doses may be higher than the threshold dose to induce serum iron rebound.

In some embodiments, the initial dose of the hepcidin or hepcidin analogue is sufficient to measurably reduce serum iron concentration, and can be from about 0.1 mg to about 40 mg, preferably from about 1 mg to about 30 mg (e.g., from about 5 mg to about 30 mg, from about 10 mg to about 30 mg, from about 20 mg to about 30 mg, from about 1 mg to about 20 mg, from about 5 mg to about 20 mg, from about 10 mg to about 20 mg, from about 1 mg to about 10 mg, from about 5 mg to about 10 mg). In some embodiments, the initial dose of the hepcidin or hepcidin analogue is about 0.1 mg, 0.5 mg, 1 mg, 2 mg, 2.5 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg or 40 mg.

In some embodiments, the additional dose or doses of the hepcidin or hepcidin analogue are sufficient to measurably reduce serum iron concentration, and can be from about 0.1 mg to about 40 mg, such as from about 1 mg to about 30 mg (e.g., from about 5 mg to about 30 mg, from about 10 mg to about 30 mg, from about 20 mg to about 30 mg, from about 1 mg to about 20 mg, from about 5 mg to about 20 mg, from about 10 mg to about 20 mg, from about 1 mg to about 10 mg, from about 5 mg to about 10 mg). In some embodiments, the additional dose or doses of the hepcidin or hepcidin analogue are about 0.1 mg, 0.5 mg, 1 mg, 2 mg, 2.5 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg or 40 mg.

The additional doses described herein may be the same, higher, or lower than the initial dose, and/or the frequency of administration may be increased or reduced, e.g., to achieve a desired effect on the patient's serum iron level or other therapeutic parameter without inducing a rebound effect. Methods described herein may include increasing the dose of the hepcidin or hepcidin analogue from one dose to a subsequent dose. Methods described herein may include decreasing the dose of the hepcidin or hepcidin analogue from one dose to a subsequent dose. Methods described herein may include increasing the frequency of administering the hepcidin or hepcidin analogue (e.g., from biweekly to weekly, or weekly to semi-weekly). Methods described herein may include decreasing the frequency of administering the hepcidin or hepcidin analogue (e.g., from semi-weekly to weekly, or from weekly to biweekly). In some embodiments, the method further comprises measuring the patient's serum iron level. In some embodiments, the method comprises adjusting or titrating the dose of administration in response to a subject's serum iron measurement.

In some embodiments, the amount of hepcidin or hepcidin analogue is such that the subject's transferrin saturation level is reduced to between 15% and 50% (e.g., between 20% and 50%, between 20% and 40%). The hepcidin or hepcidin analogue may be administered (e.g., through subcutaneous injection) in a in a single dose or in multiple doses over a period of time (e.g., once a day, once every 2 days, once every 3 days, once every 4 days, once every 5 days, once every 6 days, once every 7 days, once every 8 days, once every nine days, once every 10 days, once every 11 days, once every 12 days, once every 13 days, or once every 14 days). In some embodiments, the hepcidin or hepcidin analogue is administered twice a week, once a week, once every 10 days or once every two weeks. In some embodiments, the amount of hepcidin or hepcidin analogue is administered as necessary (e.g., when the subject experiences above-normal serum iron concentrations or above normal transferrin saturation levels (e.g., above 50%, above 60%, above 70%, above 80%, above 90% or above 95% transferrin saturation)). In some embodiments, the amount of hepcidin or hepcidin analogue is administered each time the hepcidin or hepcidin analogue is administered. In some embodiments, the hepcidin or hepcidin analogue is a mini-hepcidin. In some embodiments, the hepcidin or hepcidin analogue is administered in an amount sufficient to increase the level of hepcidin in the subject, reduce the serum ferritin concentration in the subject, reduce the total body iron level in the subject, reduce the serum iron concentration in the subject, and/or reduce the transferrin saturation in the subject. In some embodiments, the hepcidin administered is at least 80%, 85%, 90%, 91%, 92%, 93%, 94%, 95%, 96%, 97%, 98% or 99% monomer. In some embodiments, the hepcidin administered is no more than 10%, 9%, 8%, 7%, 6%, 5%, 4% 3%, 2% or 1% aggregated.

In some embodiments, provided herein are formulations comprising an amount of hepcidin or a hepcidin analogue sufficient to reduce the serum iron concentration in the subject without inducing a serum iron level rebound following treatment. In some embodiments, the amount of hepcidin or hepcidin analogue is between about 0.1 mg and about 40 mg. In some embodiments, the amount of hepcidin or hepcidin analogue is between 1 mg and about 30 mg (e.g., between about 5 mg and about 30 mg, between about 10 mg and about 30 mg, between about 20 mg and about 30 mg, between about 1 mg and about 20 mg, between about 5 mg and about 20 mg, between about 10 mg and about 20 mg, between about 1 mg and about 10 mg, between about 5 mg and about 10 mg). In some embodiments, the amount of hepcidin or hepcidin analogue is about 0.1 mg, 0.5 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg or 40 mg. In some embodiments, the hepcidin in the formulation is at least 80%, 85%, 90%, 91%, 92%, 93%, 94%, 95%, 96%, 97%, 98% or 99% monomer. In some embodiments, the hepcidin administered is no more than 10%, 9%, 8%, 7%, 6%, 5%, 4% 3%, 2% or 1% aggregated. In some embodiments, the hepcidin or hepcidin analogue is a mini-hepcidin. In some embodiments, the formulation comprises hepcidin or hepcidin analogue in an amount sufficient to increase the level of hepcidin in the subject, reduce the serum ferritin concentration in the subject, reduce the total body iron level in the subject, reduce the serum iron concentration in the subject, and/or reduce the transferrin saturation in the subject.

In some embodiments, the subject treated according to the methods provided herein has or is at risk of iron overload. In some embodiments, the subject has a transferrin saturation level of at least 50%, 60%, 70%, 80%, 90% or 95%. In some embodiments, the subject treated according to the methods provided herein has elevated serum iron levels. In some embodiments, the subject has β-thalassemia, hemochromatosis, sickle cell disease, or anemia, such as refractory anemia, hemolytic anemia, hemoglobinopathy, sideroblastic anemia, an anemia associated with myelodysplastic syndrome (MDS), or a congenital anemia. In some embodiments, the subject has liver disease (such as liver cancer), cardiomyopathy, or diabetes. The subject may have a bacterial (e.g., Escherichia coli, Neisseria cinerea, Neisseria gonorrhoeae, Staphylococcus epidermidis, Staphylococcus aureus, or Streptococcus agalactiae), viral (e.g., hepatitis B, hepatitis C, or dengue virus), fungal (e.g., Candida albicans), or protist (e.g., Trypanosoma cruzi, Plasmodium (e.g., P. falciparum, P. vivax, P. ovale, or P. malariae), Trypanosoma brucei (such as T. brucei gambiense or T. brucei rhodesiense), or Leishmania) infection. In some embodiments, the subject has received a blood transfusion (e.g., within the previous week, within the previous 48 hours, etc.). In some embodiments, the subject has transfusional iron overload.

### BRIEF DESCRIPTION OF DRAWINGS

FIG. 1 shows the serum iron percent change for subjects administered 1 mg, 5 mg, 10 mg and 20 mg doses of hepcidin.

FIG. 2 shows the percent change in serum iron through day 7 of subjects administered a 1 mg dose, a 5 mg dose, a 10 mg dose, a 20 mg dose and a 30 mg dose of hepcidin.

### DETAILED DESCRIPTION

### General

As described herein, administration of a sufficient dose of hepcidin to a subject results in reduced serum iron level in the subject and the treatment of iron overload. However, surprisingly, administration of hepcidin to the subject above a certain threshold can produce a “rebound effect”, such that, following an initial drop, the serum iron levels in the subject rise to above-baseline levels following treatment (e.g., within 30 hours of treatment, within 48 hours of treatment, within 72 hours of treatment, within 96 hours of treatment, or within about 30 to 96 hours of treatment). Provided herein are methods and compositions related to the administration of hepcidin or hepcidin analogues to a subject at an amount that sufficient to lower the subject's serum iron level but below a level that would induce serum iron level rebound following treatment. In some embodiments, the amount of hepcidin or hepcidin analogue is such that the subject's transferrin saturation level is reduced to between 15% and 50% (e.g., between 20% and 50%, between 20% and 40%).

In certain embodiments, the methods provided herein include administering to the subject hepcidin or a hepcidin analogue at an amount sufficient to reduce the serum iron concentration in the subject without inducing a serum iron level rebound following treatment. In some embodiments, provided herein are formulations comprising an amount of hepcidin or a hepcidin analogue sufficient to reduce the serum iron concentration in the subject without inducing a serum iron level rebound following treatment. In some embodiments, the amount of hepcidin or hepcidin analogue is between about 0.1 mg and 50 mg, between about 1 mg and about 30 mg, between about 5 mg and about 30 mg, between about 10 mg and about 30 mg, between about 20 mg and about 30 mg, between about 1 mg and about 20 mg, between about 5 mg and about 20 mg, between about 10 mg and about 20 mg, between about 1 mg and about 10 mg, or between about 5 mg and about 10 mg. In some embodiments, the amount of hepcidin or hepcidin analogue is about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 1.5 mg, about 2 mg, about 2.5 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg or about 40 mg.

### Definitions

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

As used herein, the term “about” and “approximately” shall generally mean an acceptable degree of error for the quantity measured given the nature or precision of the measurements. Typically, exemplary degrees of error are within 20%, preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

As used herein, the term “administering” means providing a pharmaceutical agent or composition to a subject, and includes, but is not limited to, administering by a medical professional and self-administering. Such an agent, for example, may be hepcidin or a hepcidin analogue.

As used herein, the phrase “pharmaceutically acceptable” refers to those agents, compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

As used herein, a therapeutic that “prevents” a condition (e.g., iron overload) refers to a compound that, when administered to a statistical sample prior to the onset of the disorder or condition, reduces the occurrence of the disorder or condition in the treated sample relative to an untreated control sample, or delays the onset or reduces the severity of one or more symptoms of the disorder or condition relative to the untreated control sample.

In certain embodiments, agents of the invention may be used alone or conjointly administered with another type of therapeutic agent. As used herein, the phrase “conjoint administration” refers to any form of administration of two or more different therapeutic agents such that the second agent is administered while the previously administered therapeutic agent is still effective in the body (e.g., the two agents are simultaneously effective in the subject, which may include synergistic effects of the two agents). For example, the different therapeutic agents can be administered either in the same formulation or in separate formulations, either concomitantly or sequentially. In certain embodiments, the different therapeutic agents can be administered within about one hour, about 12 hours, about 24 hours, about 36 hours, about 48 hours, about 72 hours, or about a week of one another. Thus, a subject who receives such treatment can benefit from a combined effect of different therapeutic agents.

As used herein, “serum iron rebound” or “iron rebound” refers to an increase in serum iron concentration to above-normal or above-baseline levels in a subject following an initial hepcidin-induced decrease in serum iron concentration (e.g., within 30 hours of administering hepcidin or a hepcidin analog, within 48 hours of administering hepcidin or a hepcidin analog, within 72 hours of hepcidin or a hepcidin analog, or within 96 hours of hepcidin or a hepcidin analog). As disclosed herein, serum iron rebound occurs after a subject has been administered an amount of hepcidin or a hepcidin analogue above a threshold level. In some embodiments, serum iron rebound is reflected by increased serum iron concentration in a subject following hepcidin or hepcidin analogue administration (e.g., within 48 hours of administration, within 72 hours of administration, or within 96 hours of administration) relative to the subject's serum iron concentration prior to hepcidin or hepcidin analogue administration. For example, in certain embodiments, serum iron rebound is indicated when the serum iron concentration in a subject following hepcidin or hepcidin analogue administration is at least 100%, 105%, 110%, 115%, 120%, 125%, 130%, 140%, 150%, 160%, 170%, 180%, 190%, or 200% of the subject's serum iron concentration before hepcidin administration.

As used herein, the term “subject” means a human or non-human animal selected for treatment or therapy.

The phrases “therapeutically-effective amount” and “effective amount” as used herein means the amount of an agent which is effective for producing the desired therapeutic effect in at least a sub-population of cells in a subject at a reasonable benefit/risk ratio applicable to any medical treatment.

“Treating” a disease in a subject or “treating” a subject having a disease refers to subjecting the subject to a pharmaceutical treatment, e.g., the administration of a drug, such that at least one symptom of the disease is decreased or prevented from worsening.

### Hepcidin and Hepcidin Analogues

Hepcidin is a 25-amino acid peptide with the amino acid sequence set forth in SEQ ID NO:1.

SEQ ID NO: 1
DTHFPICIFCCGCCHRSKCGMCCKT

The hepcidin peptide is a cleavage product of a larger protein, and the cell membrane protein furin can convert an extracellular hepcidin precursor protein into hepcidin peptide. The term “hepcidin or hepcidin analogue” as used herein may refer to a peptide comprising the sequence set forth in SEQ ID NO:1, including peptides that are longer than 25 amino acids, such as peptides consisting of 26 to 100 amino acids, as well as any variant of the hepcidin peptide that retains hepcidin function. For example, conservative amino acid substitutions, additions, and deletions may be made to SEQ ID NO:1 without significantly affecting the function of hepcidin. Thus, the term “hepcidin or hepcidin analogue” may refer to a peptide comprising an amino acid sequence having at least 60%, 64%, 68%, 72%, 76% 80%, 84%, 88%, 92%, 96%, or 100% sequence homology or identity with the amino acid sequence set forth in SEQ ID NO:1 that retains hepcidin activity. Sequence homology or identity may be determined using any suitable sequence alignment program, such as Protein Blast (blastp) or Clustal (e.g., ClustalV, ClustalW, ClustalX, or Clustal Omega), e.g., using default parameters, such as default weights for gap openings and gap extensions. The term “hepcidin or hepcidin analogue” may refer to a peptide comprising an amino acid sequence that is identical to the sequence set forth in SEQ ID NO:1 except that 1, 2, 3, 4, 5, 6, 7, 8, 9, or 10 amino acids of SEQ ID NO:1 are substituted with different amino acids, e.g., that preferably retains the disulfide-bonding of naturally occurring hepcidin. In some embodiments, hepcidin comprises a cysteine at each of the positions in which a cysteine occurs in SEQ ID NO:1.

In some embodiments, the hepcidin is at least 80% monomer. For example, in some embodiments, the hepcidin is at least 81%, 82%, 83%, 84%, 85%, 86%, 87%, 88%, 89%, 90%, 91%, 92%, 93%, 94%, 95%, 96%, 97%, 98% or 99% monomer. In some embodiments, the hepcidin is no more than 10%, 9%, 8%, 7%, 6%, 5%, 4%, 3%, 2% or 1% aggregates (e.g., aggregates larger than dimers).

N-terminal and C-terminal residues may be deleted from the hepcidin peptide without significantly affecting its activity. Thus, in some embodiments, “hepcidin or hepcidin analogue” may include a peptide comprising the sequence set forth in SEQ ID NO:2, SEQ ID NO:3, or SEQ ID NO:4, or a peptide comprising an amino acid sequence having at least 60%, 65%, 70%, 75%, 80%, 85% 90%, 95% or 100% sequence homology or identity with the amino acid sequence set forth in SEQ ID NO:2, SEQ ID NO:3, SEQ ID NO:4, or SEQ ID NO:5.

SEQ ID NO: 2
PICIFCCGCCHRSKCGMCCKT
SEQ ID NO: 3
PICIFCCGCCHRSKCGMCC
SEQ ID NO: 4
ICIFCCGCCHRSKCGMCCKT
SEQ ID NO: 5
CIFCCGCCHRSKCGMCC

The term “hepcidin or hepcidin analogue” may refer to a peptide comprising an amino acid sequence that is identical to the sequence set forth in SEQ ID NO:2, SEQ ID NO:3, SEQ ID NO:4, or SEQ ID NO:5 except that 1, 2, 3, 4, 5, 6, 7, 8, 9, or 10 amino acids of SEQ ID NO:2, SEQ ID NO:3, SEQ ID NO:4, or SEQ ID NO:5 are substituted with different amino acids. In some embodiments, hepcidin or the hepcidin analogue comprises a cysteine at each of the positions in which a cysteine occurs in SEQ ID NO:2, SEQ ID NO:3, SEQ ID NO:4, or SEQ ID NO:5.

In some embodiments, the term “hepcidin or hepcidin analogue” refers to a peptide comprising an amino acid sequence that is identical to the sequence set forth in SEQ ID NO:6, SEQ ID NO:7, SEQ ID NO:8, SEQ ID NO:9, or SEQ ID NO:10. In SEQ ID NO:6, SEQ ID NO:7, SEQ ID NO:8, SEQ ID NO:9, or SEQ ID NO:10, the amino acids labeled “X” may be any amino acid, including naturally occurring and non-naturally occurring amino acids. In some embodiments, each of the amino acids labeled “X” is a naturally occurring amino acid.

SEQ ID NO: 6
XXHXPXCXXCCGCCHRSKCGMCCXX
SEQ ID NO: 7
PXCXXCCGCCHRSKCGMCCKX
SEQ ID NO: 8
PXCXXCCGCCHRSKCGMCC
SEQ ID NO: 9
XCXXCCGCCHRXXCGXCCKX
SEQ ID NO: 10
CXXCCGCCHRXXCGXCC

In some embodiments, “hepcidin or hepcidin analogue” is a molecule that specifically binds to ferroportin and/or iron (e.g., an iron cation). Hepcidin or a hepcidin analogue may comprise 1, 2, 3, or 4 disulfide bonds. In some embodiments, hepcidin comprises four disulfide bonds. In some embodiments, each of the four disulfide bonds is an intramolecular disulfide bond. In some embodiments, each of the eight cysteines of SEQ ID NO:1, 2, 3, 4, 5, 6, 7, 8, 9, or 10 participates in one of four intramolecular disulfide bonds with another one of the eight cysteines. In some embodiments, “hepcidin or hepcidin analogue” may include a peptide comprising the sequence set forth in SEQ ID NO:6, SEQ ID NO:7, SEQ ID NO:8, SEQ ID NO:9, or SEQ ID NO:10, or a peptide comprising an amino acid sequence having at least 60%, 65%, 70%, 75%, 80%, 85% 90%, 95% or 100% sequence homology or identity with the amino acid sequence set forth in SEQ ID NO:6, SEQ ID NO:7, SEQ ID NO:8, SEQ ID NO:9, or SEQ ID NO:10.

In some embodiments, hepcidin or the hepcidin analogue has about 10% to 1000% of the activity of a 25 amino acid long peptide comprising the amino acid sequence set forth in SEQ ID NO:1, i.e., wherein the 25 amino acid long peptide comprises the four intramolecular disulfide bonds found in native human hepcidin. For example, hepcidin may have about 50% to about 200% of the activity of a 25 amino acid long peptide comprising the amino acid sequence set forth in SEQ ID NO:1 (i.e., wherein the 25 amino acid long peptide comprises the four intramolecular disulfide bonds found in native human hepcidin), such as about 75% to about 150% of the activity, about 80% to about 120% of the activity, about 90% to about 110% of the activity, or about 95% to about 105% of the activity. The term “activity” may refer to the ability of hepcidin or the hepcidin analogue to specifically bind to ferroportin, e.g., thereby inhibiting the transport of intracellular iron into the extracellular space, inhibiting the absorption of dietary iron, and/or reducing serum iron concentration. Activity may refer to the ability of hepcidin or the hepcidin analogue to inhibit the transport of intracellular iron into the extracellular space. Activity may refer to the ability of hepcidin to inhibit the absorption of dietary iron. Activity may refer to the ability of hepcidin or hepcidin analogue to reduce excess iron in vivo. Activity may refer to the ability of hepcidin or hepcidin analogue to reduce serum iron concentration in vivo. Because of the different potencies of various hepcidin analogues, unless otherwise specified herein, a specific dose of hepcidin or a hepcidin analogue should be understood as that dose of hepcidin or a dose of a hepcidin analogue that achieves the same therapeutic effect (e.g., the same reduction in serum iron levels) as the specified dose of hepcidin.

In some embodiments, hepcidin or the hepcidin analogue may refer to a mini-hepcidin, a modified hepcidin, or a hepcidin mimetic peptide. The structure of the bioactive 25-amino acid form of hepcidin is a hairpin with 8 cysteines that form 4 disulfide bonds as described by Jordan et al. (2009) J Biol Chem 284:24155-67, which is hereby incorporated by reference in its entirety. The N-terminal region has been shown to be required for iron-regulatory function, and deletion of 5 N-terminal amino acid residues results in a loss of iron-regulatory function. This finding has resulted in the design of drug-like hepcidin mimetic peptides (Preza et al., Clin Invest. 2011; 121(12):4880-4888), which is hereby incorporated by reference in its entirety. Hepcidin mimetic peptides or modified hepcidins are peptides that exert the function of reducing serum iron concentration as bioactive hepcidin.

For the purposes of this application, the terms mini-hepcidin, a modified hepcidin, or a hepcidin mimetic peptide may be used interchangeably. Mini-hepcidins, a modified hepcidin, and hepcidin mimetic peptides are disclosed in US. Pat. No. 9,315,545, 9,328,140, and 8,435,941, and in U.S. Publication number US2015284429, each of which is hereby incorporated by reference, in particular for their disclosure of compounds that share one or more activities with hepcidin.

A mini-hepcidin may have the structure of Formula I:

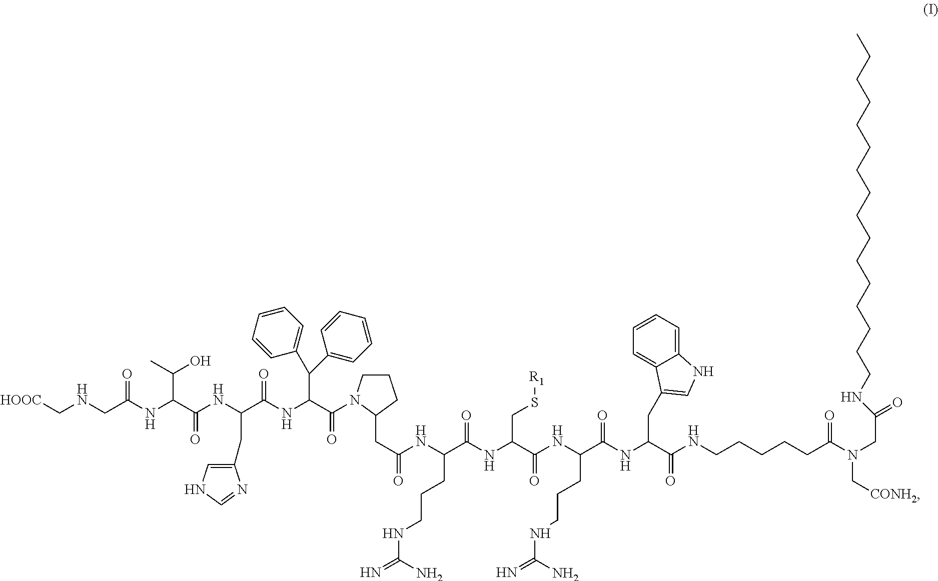

wherein R₁ is, —S—Z₁; —Z₂, —SH, —C(═O)—Z₃ or —S—C(═O)—Z₃,
Z₁ is substituted or unsubstituted C₁-C₁₈ alkyl or C₁-C₁₈ alkenyl, wherein the C₁-C₁₈ alkyl or C₁-C₁₈ is alkenyl is branched or unbranched or Z₁ is an electron withdrawing or donating group;
Z₂ is substituted or unsubstituted C₁-C₁₈ alkyl or C₁-C₁₈ alkenyl, wherein the C₁-C₁₈ alkyl or C₁-C₁₈ alkenyl is branched or unbranched or Z₂ is an electron withdrawing or donating group;
Z₃ is substituted or unsubstituted C₁-C₁₈ alkyl or C₁-C₁₈ alkenyl, wherein the C₁-C₁₈ alkyl or C₁-C₁₈ alkenyl is branched or unbranched or Z₃ is an electron withdrawing or donating group.

A mini-hepcidin may have the structure of any one of Formulas II-IV:

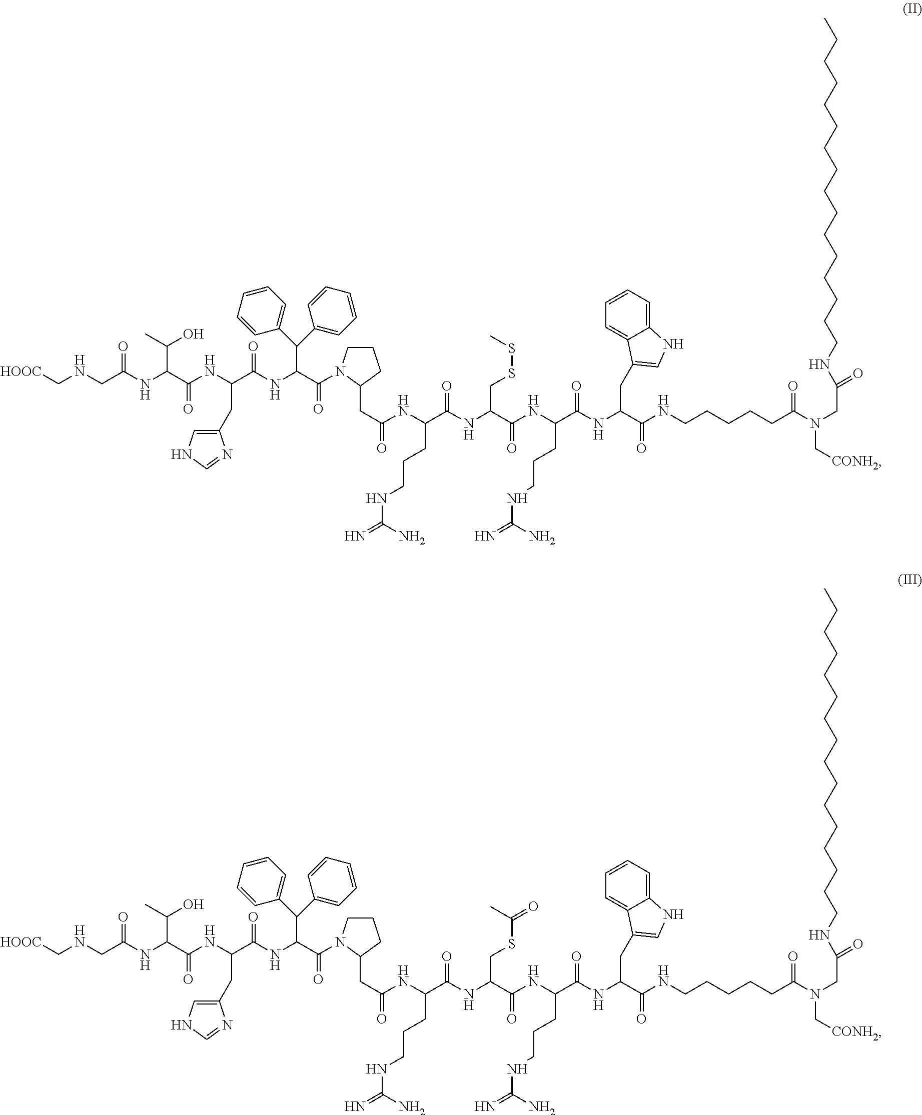 [SMILES: CCCCCCCCCCCCCCCCNC(=O)CN(CC(N)=O)C(=O)CCCCCNC(=O)C(Cc1c[nH]c2ccccc12)NC(=O)C(CCCNC(=N)N)NC(=O)C(CSSC)NC(=O)C(CCCNC(=N)N)NC(=O)CC1CCCN1C(=O)C(NC(=O)C(Cc1c[nH]cn1)NC(=O)C(NC(=O)CNCC(=O)O)C(C)O)C(c1ccccc1)c1ccccc1 ; CCCCCCCCCCCCCCCCNC(=O)CN(CC(N)=O)C(=O)CCCCCNC(=O)C(Cc1c[nH]c2ccccc12)NC(=O)C(CCCNC(=N)N)NC(=O)C(CSC(C)=O)NC(=O)C(CCCNC(=N)N)NC(=O)CC1CCCN1C(=O)C(NC(=O)C(Cc1c[nH]cn1)NC(=O)C(NC(=O)CNCC(=O)O)C(C)O)C(c1ccccc1)c1ccccc1]

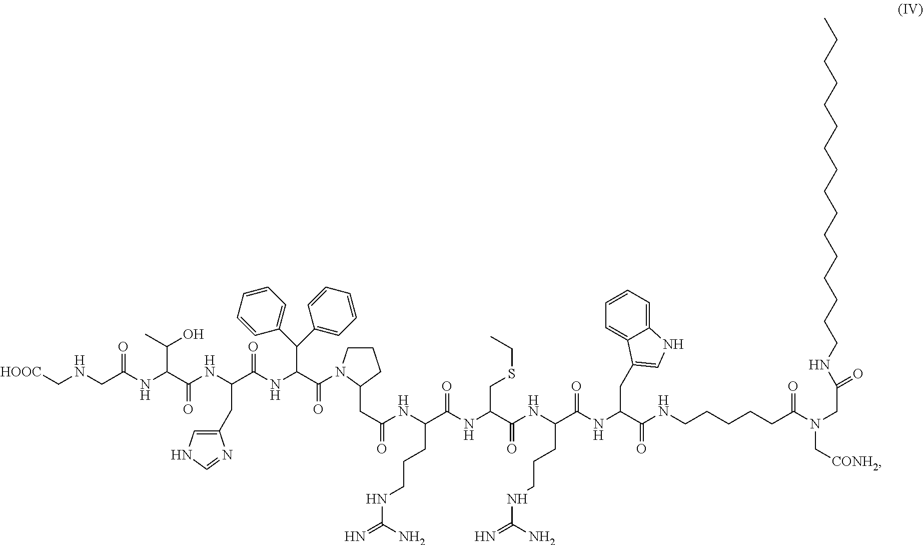 [SMILES: CCCCCCCCCCCCCCCCNC(=O)CN(CC(N)=O)C(=O)CCCCCNC(=O)C(Cc1c[nH]c2ccccc12)NC(=O)C(CCCNC(=N)N)NC(=O)C(CSCC)NC(=O)C(CCCNC(=N)N)NC(=O)CC1CCCN1C(=O)C(NC(=O)C(Cc1c[nH]cn1)NC(=O)C(NC(=O)CNCC(=O)O)C(C)O)C(c1ccccc1)c1ccccc1]

A mini-hepcidin may have the structure of Formula V:

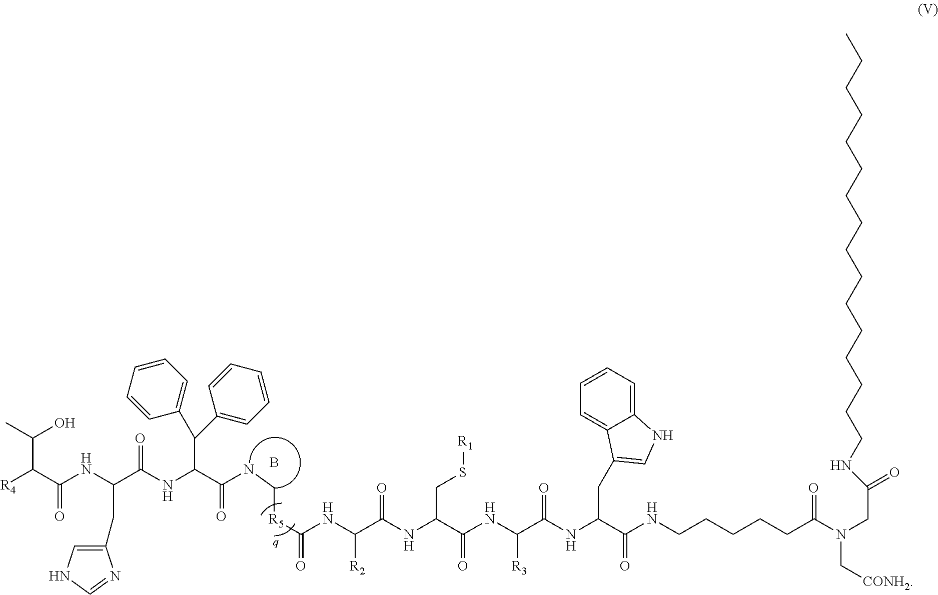

wherein:
R₁ is, H, —S—Z₁; —Z₂, —SH, —C(═O)—Z₃, or —S—C(═O)—Z₃,
R₂ and R₃ are each, independently, optionally substituted C₄-C₇ alkyl,

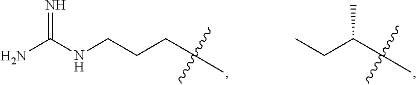

D-Arg, D-Ile, Leu, D-Leu, Thr, D-Thr, Lys, D-Lys, Val, D-Val, D-Nω,ω-dimethyl-arginine, L-Nω, ω-dimethyl-arginine, D-homoarginine, L-homoarginine, D-norarginine, L-norarginine, citrulline, a modified Arg wherein the guanidinium group is modified or substituted, norleucine, norvaline, beta homo-Ile, 1-aminocyclohexane-1-carboxylic acid, N-Me-Arg, N-Me-Ile;
R₄ is Ida, Asp, Acetyl-Asp, (methylamino)pentanedioic acid, Acetyl-Gly-Ida, or Acetyl-Gly-Asp or a derivative thereof to remove its negative charge above pH 4;
R₅ is CR₆R₇, aryl or heteroaryl;
B is absent or forms a 5-7 membered ring; and
q is 0-6, wherein when R₅ aryl or heteroaryl q is 1 and B is absent;
Z₁ is substituted or unsubstituted C₁-C₁₈ alkyl, wherein the C₁-C₁₈ alkyl is branched or unbranched;
Z2 is substituted or unsubstituted C₁-C₁₈ alkyl, wherein the C₁-C₁₈ alkyl is branched or unbranched;
Z3 is substituted or unsubstituted C₁-C₁₈ alkyl, wherein the C₁-C₁₈ alkyl is branched or unbranched;
R₆ and R₇ are each, independently, H, halo, optionally substituted C₁-C₃ alkyl, or haloalkyl, provided that when Ri is H, the compound does not have the structure of Formula XVI.

A mini-hepcidin may have the structure of any one of Formulas VI-VIII:

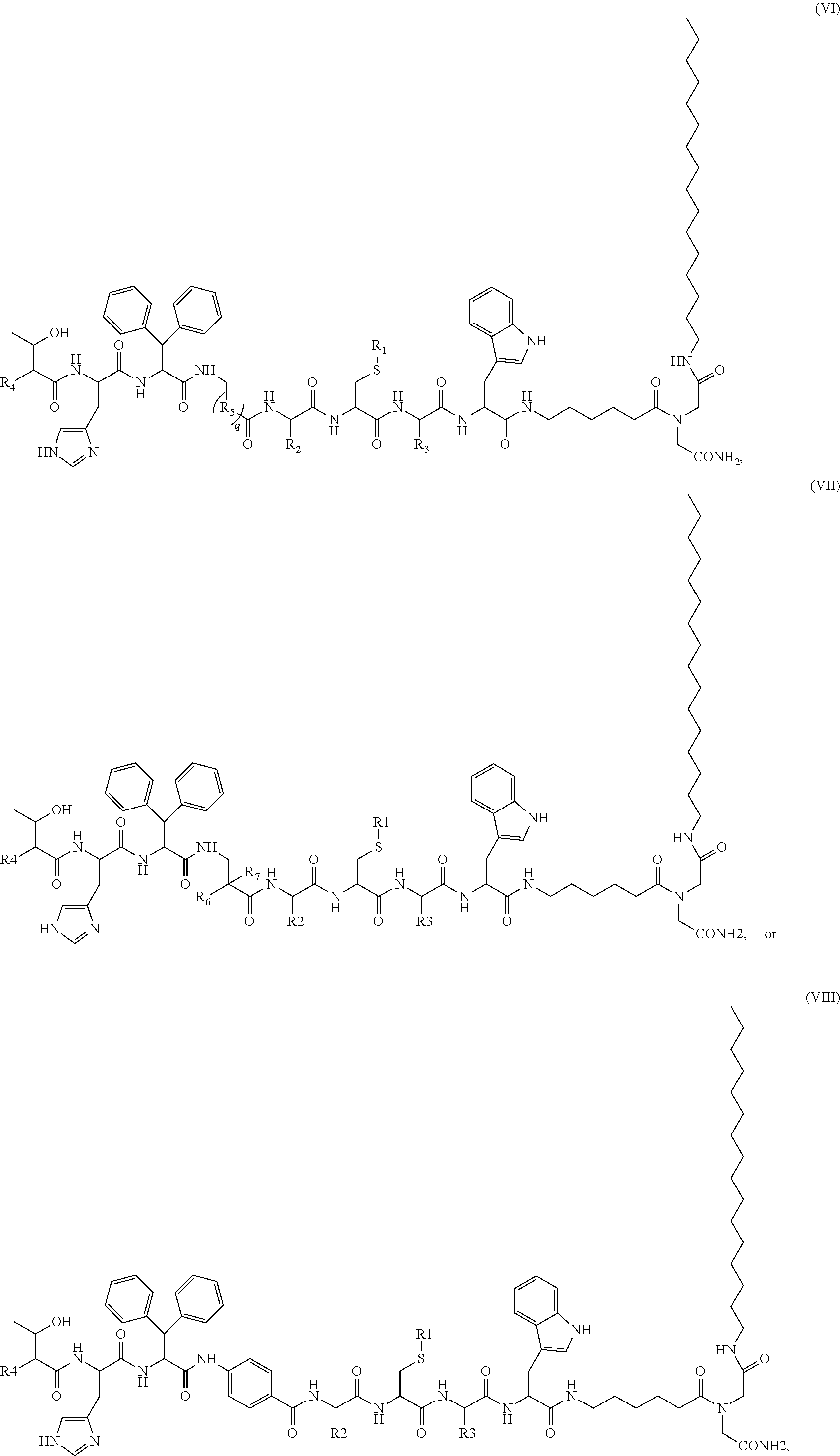

wherein the variables are defined as for Formula V.

A mini-hepcidin may have the structure of Formula IX:

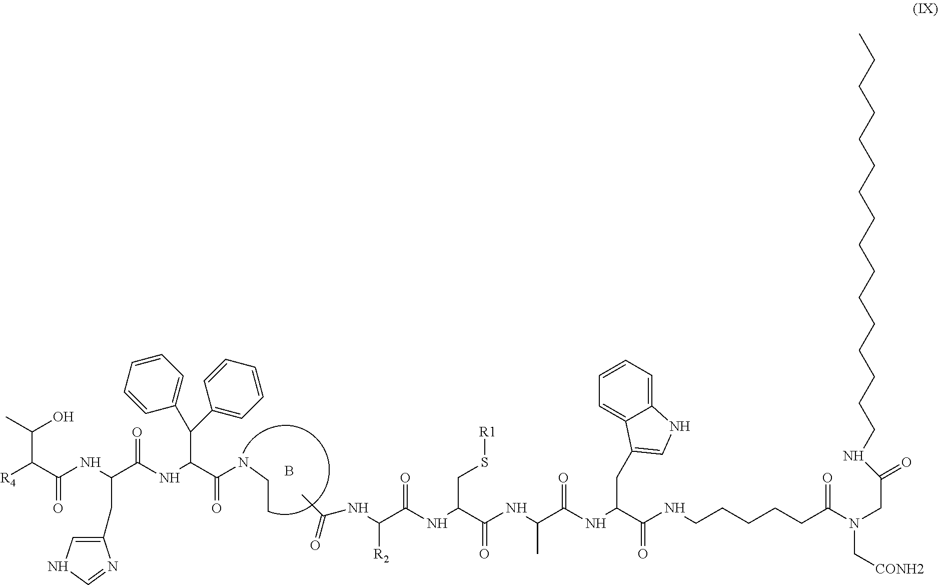

wherein R₁ is H, —S—Z₁, —Z₂, —SH, —S—C(═O)—Z₃,
R₂ and R₃ are each, independently, optionally substituted C₄-C₇ alkyl,

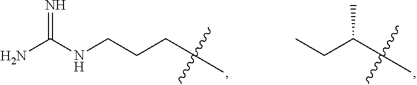

D-Arg, D-Ile, Leu, D-Leu, Thr, D-Thr, Lys, D-Lys, Val, D-Val, D-Nω,ω-dimethyl-arginine, L-Nω, ω-dimethyl-arginine, D-homoarginine, L-homoarginine, D-norarginine, L-norarginine, citrulline, a modified Arg wherein the guanidinium group is modified or substituted, norleucine, norvaline, beta homo-Ile, 1-aminocyclohexane-1-carboxylic acid, N-Me-Arg, N-Me-Ile;
R₄ is Ida, Asp, Acetyl-Asp, (methylamino)pentanedioic acid, Acetyl-Gly-Ida, or Acetyl-Gly-Asp or a derivative thereof to remove its negative charge above pH 4;
B is absent or forms a 5-7 membered ring;
Z₁ is substituted or unsubstituted C₁-C₁₈ alkyl, wherein the C₁-C₁₈ alkyl is branched or unbranched;
Z₂ is substituted or unsubstituted C₁-C₁₈ alkyl, wherein the C₁-C₁₈ alkyl is branched or unbranched; and
Z₃ is substituted or unsubstituted C₁-C₁₈ alkyl, wherein the C₁-C₁₈ alkyl is branched or unbranched;
provided that when R₁ is H, the compound does not have the structure of Formula XVI.

A mini-hepcidin may have the structure of Formula X:

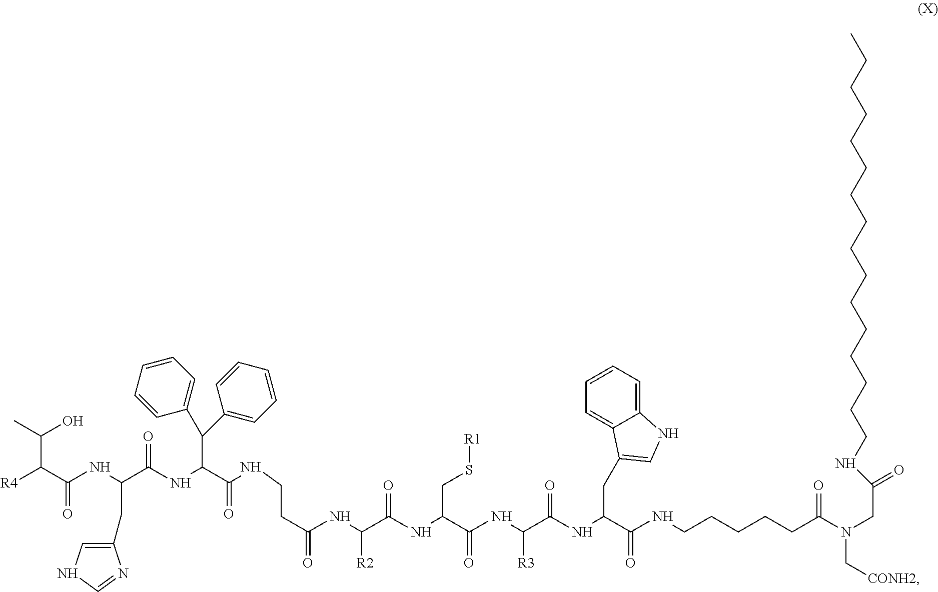

wherein the variables are defined as for Formula IX.

A mini-hepcidin may have the structure of Formula XI:

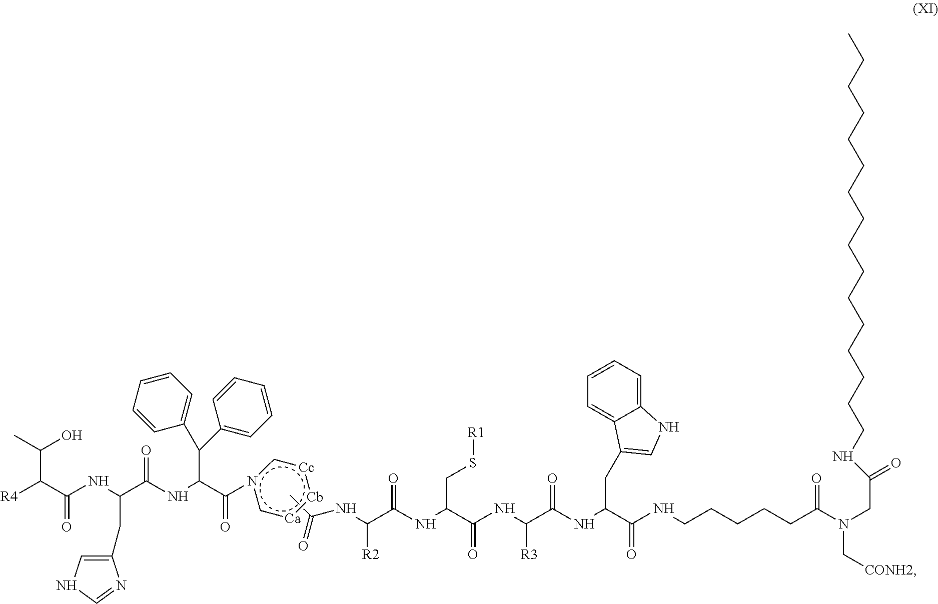

wherein the carbonyl forms a bond with the 6-membered ring at Ca, Cb, or Cc and with the variables as defined for Formula IX.

A mini-hepcidin may have the structure of Formula XII:

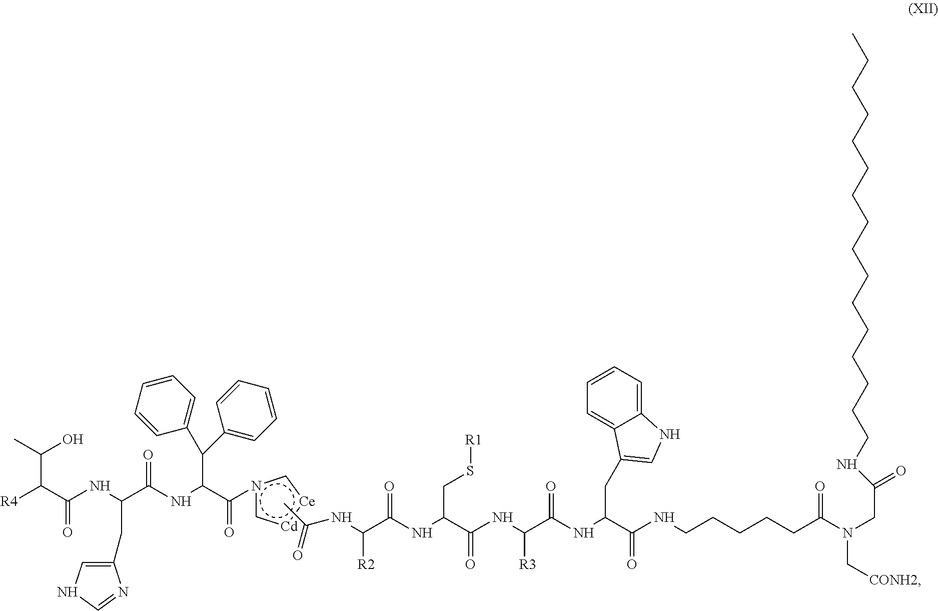

wherein the carbonyl forms a bond with the 5-membered ring at Ca or Ce and with the variables as defined for Formula IX.

A mini-hepcidin may have the structure of Formula

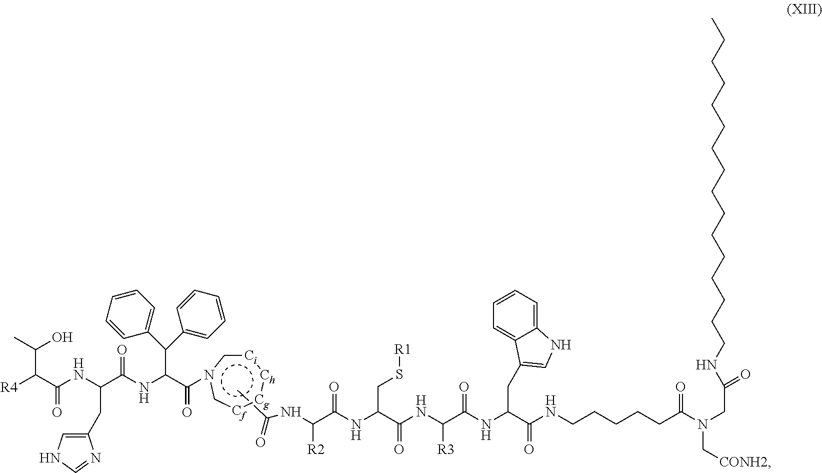

wherein the bond from the carbonyl forms a bond with the 7-membered ring at C_{f}, C_{g}, Cₕ, or C₁ and with the variables as defined for Formula IX.

A mini-hepcidin may have the structure of Formula XIV:

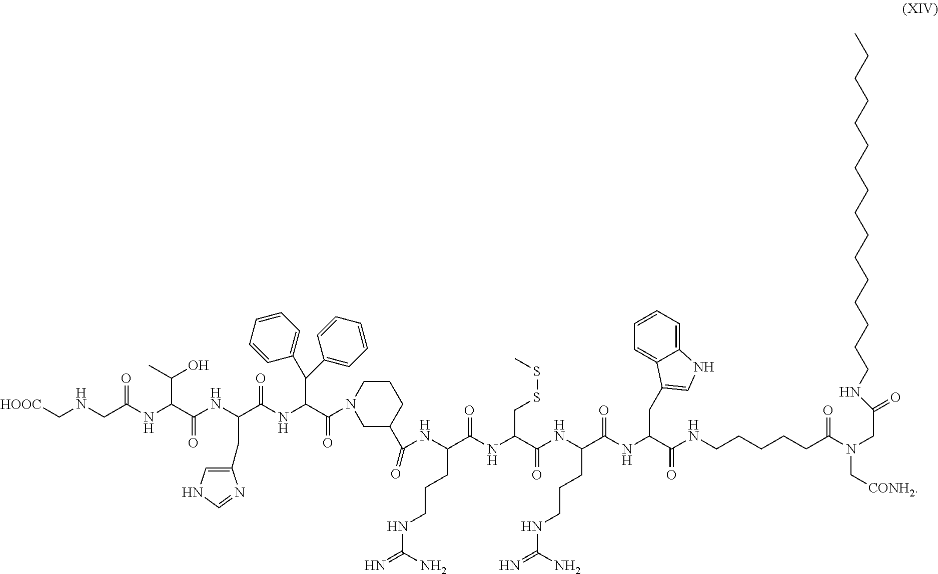 [SMILES: CCCCCCCCCCCCCCCCNC(=O)CN(CC(N)=O)C(=O)CCCCCNC(=O)C(Cc1c[nH]c2ccccc12)NC(=O)C(CCCNC(=N)N)NC(=O)C(CSSC)NC(=O)C(CCCNC(=N)N)NC(=O)C1CCCN(C(=O)C(NC(=O)C(Cc2c[nH]cn2)NC(=O)C(NC(=O)CNCC(=O)O)C(C)O)C(c2ccccc2)c2ccccc2)C1]

A mini-hepcidin may have the structure of Formula XV:

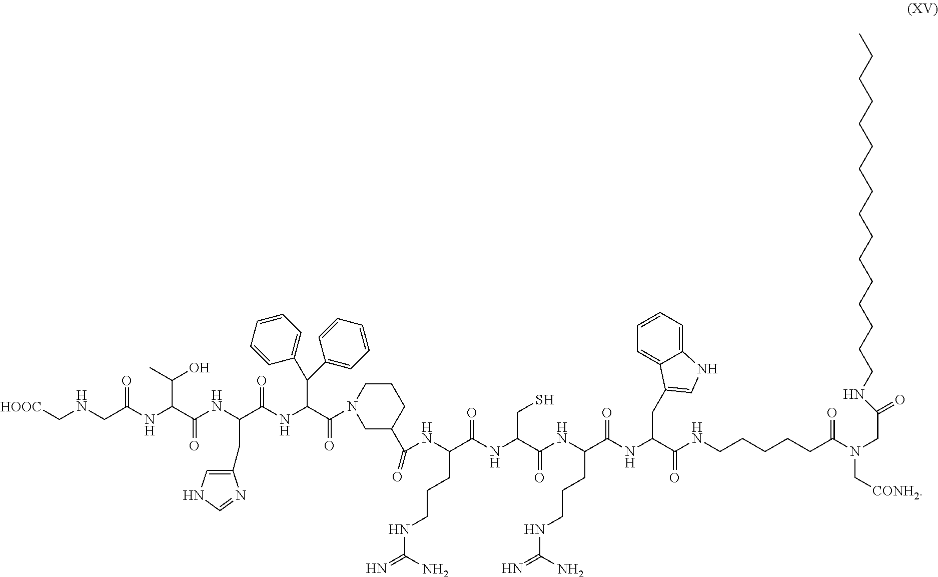 [SMILES: CCCCCCCCCCCCCCCCNC(=O)CN(CC(N)=O)C(=O)CCCCCNC(=O)C(Cc1c[nH]c2ccccc12)NC(=O)C(CCCNC(=N)N)NC(=O)C(CS)NC(=O)C(CCCNC(=N)N)NC(=O)C1CCCN(C(=O)C(NC(=O)C(Cc2c[nH]cn2)NC(=O)C(NC(=O)CNCC(=O)O)C(C)O)C(c2ccccc2)c2ccccc2)C1]

A mini-hepcidin may have the structure of Formula P₁-P₂-P₃-P₄-P₅-P₆-P₇-P₈-P₉-P₁₀ or P₁₀-P₉-P₈-P₇-P₆-P₅-P₄-P₃-P₂-P₁, wherein P₁ to P₁₀ are as defined in table 1; X₃ is Ahx-Ida(NH-PAL)-NH₂, Ida is Iminodiacetic acid; Dpa is 3,3-diphenyl-L-alanine; bhPro is beta-homoproline; Npc is L-nipecotic acid; isoNpc is isonipecotic acid; and bAla is beta-alanine.

TABLE 1

| P₁ | P₂ | P₃ | P₄ | P₅ | P₆ | P₇ | P₈ | P₉ | P₁₀ |
|---|---|---|---|---|---|---|---|---|---|
| Ida | Thr | His | Dpa | bhPro | Arg | Cys-S—CH₃ | Arg | Trp | X₃ |
| Ida | Thr | His | Dpa | bhPro | Arg | Cys-C(═O)CH₃ | Arg | Trp | X₃ |
| Ida | Thr | His | Dpa | bhPro | Arg | Cys-CH₂—CH₃ | Arg | Trp | X₃ |
| Ida | Thr | His | Dpa | Npc | Arg | Cys-S—CH₃ | Arg | Trp | X₃ |
| Ida | Thr | His | Dpa | Npc | Arg | Cys | Arg | Trp | X₃ |
| Ida | Thr | His | Dpa | D-Npc | Arg | Cys-S—CH₃ | Arg | Trp | X₃ |
| Ida | Thr | His | Dpa | isoNpc | Arg | Cys-S—CH₃ | Arg | Trp | X₃ |
| Acetyl-Gly-Ida | Thr | His | Dpa | bhPro | Arg | Cys-S—CH₃ | Arg | Trp | X₃ |
| Ida | Thr | His | Dpa | bAla | Arg | Cys-S—CH₃ | Arg | Trp | X₃ |

A mini-hepcidin may have the structure of Formula XVI:

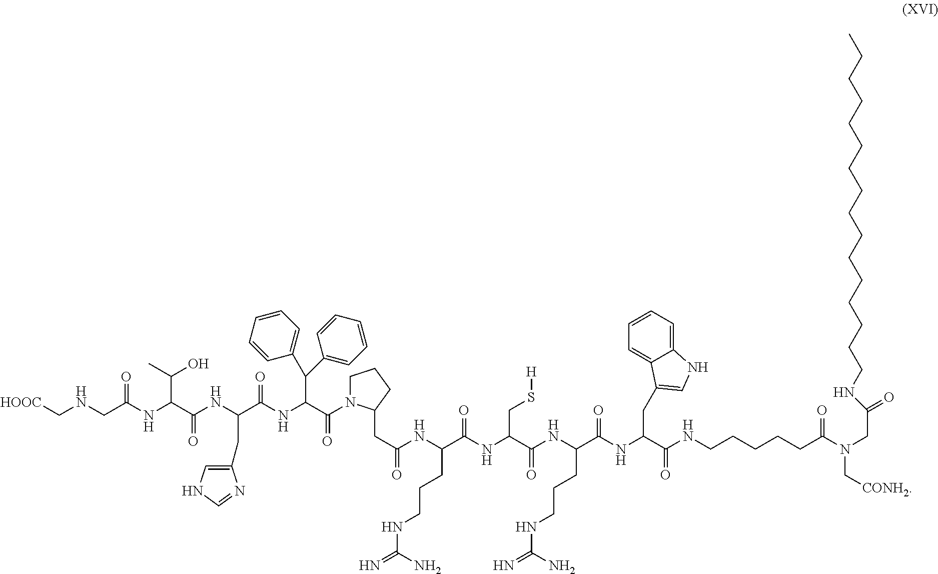 [SMILES: CCCCCCCCCCCCCCCCNC(=O)CN(CC(N)=O)C(=O)CCCCCNC(=O)C(Cc1c[nH]c2ccccc12)NC(=O)C(CCCNC(=N)N)NC(=O)C(CS)NC(=O)C(CCCNC(=N)N)NC(=O)CC1CCCN1C(=O)C(NC(=O)C(Cc1c[nH]cn1)NC(=O)C(NC(=O)CNCC(=O)O)C(C)O)C(c1ccccc1)c1ccccc1]

A mini-hepcidin may have the structure of formula A1-A2-A3-A4-A5-A6-A7-A8-A9-A10, A10-A9-A8-A7-A6-A5-A4-A3-A2-A1, wherein:
A1 is L-Asp, L-Glu, pyroglutamate, L-Gln, L-Asn, D-Asp, D-Glu, D-pyroglutamate, D-Gln, D-Asn, 3-aminopentanedioic acid, 2,2′-azanediyldiacetic acid, (methylamino)pentanedioic acid, L-Ala, D-Ala, L-Cys, D-Cys, L-Phe, D-Phe, L-Asp, D-Asp, 3,3-diphenyl-L-alanine, 3,3-diphenyl-D-alanine; and if A1 is L-Asp or D-Asp, then A2 is L-Cys or D-Cys; if A1 is L-Phe or D-Phe, then the N-terminus is optionally attached to a PEG molecule linked to chenodeoxvcholate, ursodeoxvcholate, or palmitoyl; or if A1 is 3,3-diphenyl-L-alanine or 3,3-diphenyl-D-alanine, then the N-terminus is attached to palmitoyl;
A2 is L-Thr, L-Ser, L-Val, L-Ala, D-Thr, D-Ser, D-Val, L-tert-leucine, isonipecotic acid, L-α-cyclohexylglycine, bhThr, (2S)-3-hydroxy-2-(methylamino)butanoic acid, D-Ala, L-Cys, D-Cys, L-Pro, D-Pro, or Gly;
A3 is L-His, D-His, 3,3-diphenyl-L-alanine, 3,3-diphenyl-D-alanine, or 2-aminoindane;
A4 is L-Phe, D-Phe, (S)-2-amino-4-phenylbutanoic acid, 3,3-diphenyl-L-alanine, L-biphenylalanine, (1-naphthyl)-L-alanine, (S)-3-Amino-4,4-diphenylbutanoic acid, 4-(aminomethyl)cyclohexane carboxylic acid, (S)-2-amino-3-(perfluorophenyl)propanoic acid, (S)-2-amino-4-phenylbutanoic acid, (S)-2-amino-2-(2,3-dihydro-1H-inden-2-yl)acetic acid, or cyclohexylalanine;
A5 is L-Pro, D-Pro, octahydroindole-2-carboxylic acid, L-β-homoproline, (2S,4S)-4-phenylpyrrolidine-2-carboxylic acid, (2S,5R)-5-phenylpyrrolidine-2-carboxylic acid, or (R)-2-methylindoline;
A6 is L-Ile, D-Ile, L-phenylglycine, L-α-cyclohexylglycine, 4-(aminomethyl)cyclohexane carboxylic acid, (3R)-3-amino-4-methylhexanoic acid, 1-aminocyclohexane-1-carboxylic acid, or (3R)-4-methyl-3-(methylamino)hexanoic acid;
A7 is L-Cys, D-Cys, S-t-Butylthio-L-cysteine, L-homocysteine, L-penicillamine, or D-penicillamine;
A8 is L-Ile, D-Ile, L-α-cyclohexylglycine, 3,3-diphenyl-L-alanine, (3R)-3-amino-4-methylhexanoic acid, 1-aminocyclohexane-1-carboxylic acid, or (3R)-4-methyl-3-(methylamino)hexanoic acid;
A9 is L-Phe, L-Leu, L-Ile, L-Tyr, D-Phe, D-Leu, D-Ile, (S)-2-amino-3-(perfluorophenyl)propanoic acid, N-methyl-phenylalainine, benzylamide, (S)-2-amino-4-phenylbutanoic acid, 3,3-diphenyl-L-alanine, L-biphenylalanine, (1-naphthyl)-L-alanine, (S)-3-amino-4,4-diphenylbutanoic acid, cyclohexylalanine, L-Asp, D-Asp, or cysteamide, wherein L-Phe or D-Phe are optionally linked at the N-terminus to RA, wherein RA is —CONH—CH₂—CH₂—S—, or D-Pro linked to Pro-Lys or Pro-Arg, or L-β-homoproline linked to L-Pro linked to Pro-Lys or Pro-Arg, or D-Pro linked to L-β-homoproline-Lys or L-β-homoproline-Arg; L-Asp or D-Asp are optionally linked at the n-terminus to RB, wherein RB is -(PEG 11)-GYIPEAPRDGQAYVRKDGEWVLLSTFL, or -(PEG 11)-(Gly-Pro-HydroxyPro)₁₀, (S)-2-amino-4-phenylbutanoic acid is linked to RC, wherein RC is D-Pro linked to ProLys or ProArg, or D-Pro linked to L-β-homoproline-Lys or L-β-homoproline-L-Arg;
A10 is L-Cys, L-Ser, L-Ala, D-Cys, D-Ser, or D-Ala;
the carboxy-terminal amino acid is in amide or carboxy-form;
at least one sulfhydryl amino acid is present as one of the amino acids in the sequence; and
A1, A2, A1 to A2, A10, A9 to A10, or a combination thereof are optionally absent.

A mini-hepcidin of formula A1-A2-A3-A4-A5-A6-A7-A8-A9-A10 or A10-A9-A8-A7-A6-A5-A4-A3-A2-A1 may be a cyclic peptide or a linear peptide.

For example, A1 may be L-Asp; A2, may be L-Th; A3 may be L-His; A4 may be L-Phe; A5 may be L-Pro; A6 may be L-Ile; A7 may be L-Cys, D-Cys, S-t-butylthio-L-cysteine, L-homocysteine, L-penicillamine, or D-penicillamine; A8 may be L-Ile; A9 may be L-Phe; A10 may be absent; and the C-terminus may be amidated. Alternatively, A3 may be L-His; A4 may be L-Phe; A5 may be L-Pro; A6 may be L-Ile; A7 may be L-Cys, D-Cys, S-t-butylthio-L-cysteine, L-homocysteine, L-penicillamine, or D-penicillamine; A8 may be L-Ile; A1, A2, A9, and A10 may be absent, and the C-terminus may be amidated. Alternatively, A3 may be L-His; A4 may be L-Phe; A5 may be L-Pro; A6 may be L-Ile; A7 may be L-Cys, D-Cys, S-t-butylthio-L-cysteine, L-homocysteine, L-penicillamine, or D-penicillamine; A1, A2, A8, A9, and A10 may be absent; and the C-terminus may be amidated.

A mini-hepcidin, may comprise the amino acid sequence HFPICI (SEQ ID NO:11), HFPICIF (SEQ ID NO:12), DTHFPICIDTHFPICIF (SEQ ID NO:13), DTHFPIAIFC (SEQ ID NO:14), DTHAPICIF (SEQ ID NO:15), DTHFPICIF (SEQ ID NO:16), or CDTHFPICIF (SEQ ID NO:17). The mini-hepcidin may comprise the sequence set forth in SEQ ID NO:15, for example, wherein the cysteine forms a disulfide bond with S-tertbutyl.

A mini-hepcidin may comprise the amino acid sequence D-T-H-F-P-I-(L-homocysteine)-I-F; D-T-H-F-P-I-(L-penicillamine)-I-F; D-T-H-F-P-I-(D-penicillamine)-I-F; D-(L-tert-leucine)-H-(L-phenylglycine)-(octahydroindole-2-carboxylic acid)-(L-α-cyclohexylglycine)-C-(L-α-cyclohexylglycine)-F; or D-(L-tert-leucine)-H-P-(octahydroindole-2-carboxylic acid)-(L-α-cyclohexylglycine)-C-(L-α-cyclohexylglycine)-F.

A mini-hepcidin may comprise the amino acid sequence FICIPFHTD (SEQ ID NO:18), FICIPFH (SEQ ID NO:19), R2-FICIPFHTD (SEQ ID NO:20), R3-FICIPFHTD (SEQ ID NO:21), FICIPFHTD-R6 (SEQ ID NO:22), R4-FICIPFHTD (SEQ ID NO:23), or R5-FICIPFHTD (SEQ ID NO:24), wherein each amino acid is a D amino acid; R1 is —CONH₂—CH₂—CH₂—S; R₂ is chenodeoxycholate-(PEG 11)-; R3 is ursodeoxycholate-(PEG11)-; R4 is palmitoyl-(PEG11)-; R5 is 2(palmitoyl)-diaminopropionic acid-(PEG 11)-; and R6 is (PEG 11)-GYIPEAPRDGQAYVRKDGEWVLLSTFL, wherein each amino acid of R6 is an L amino acid.

A mini-hepcidin may comprise the amino acid sequence D-T-H-((S)-2-amino-4-phenylbutanoic acid)-P-I-C-I-F; D-T-H-(3,3-diphenyl-L-alanine)-P-I-C-I-F; D-T-H-(L-biphenylalanine)-P-I-C-I-F; D-T-H-((1-naphthyl)-L-alanine)-P-I-C-I-F; D-T-H-((S)-3-amino-4,4-diphenylbutanoic acid)-P-I-C-I-F; D-T-H-F-P-I-C-I-((S)-2-amino-4-phenylbutanoic acid); D-T-H-F-P-I-C-I-(3,3-diphenyl-L-alanine); D-T-H-F-P-I-C-I-(L-biphenylalanine); D-T-H-F-P-I-C-I-((1-naphthyl)-L-alanine); D-T-H-F-P-I-C-I-((S)-3-amino-4,4-diphenylbutanoic acid); D-T-H-(3,3-diphenyl-L-alanine)-P-I-C-I-(3,3-diphenyl-L-alanine); D-(3,3-diphenyl-L-alanine)-P-I-C-I-F; D-(3,3-diphenyl-L-alanine)-P-I-C-I-(3,3-diphenyl-L-alanine); D-T-H-(3,3-diphenyl-L-alanine)-P-R-C-R-(3,3-diphenyl-L-alanine); D-T-H-(3,3-diphenyl-L-alanine)-(octahydroindole-2-carboxylic acid)-I-C-I-F; D-T-H-(3,3-diphenyl-L-alanine)-(octahydroindole-2-carboxylic acid)-I-C-I-(3,3-diphenyl-L-alanine); or D-T-H-(3,3-diphenyl-L-alanine)-P-C-C-C-(3,3-diphenyl-L-alanine).

A mini-hepcidin may comprise the amino acid sequence D-T-H-F-P-I-C-I-F-R8; D-T-H-F-P-I-C-I-F-R9; D-T-H-F-P-I-C-I-F-R10; D-T-H-F-P-I-C-I-F-R11; D-T-H-F-P-I-C-I-F-R12; D-T-H-F-P-I-C-I-F-R13; D-T-H-F-P-I-C-I-((S)-2-amino-4-phenylbutanoic acid)-R8; D-T-H-F-P-I-C-I-((S)-2-amino-4-phenylbutanoic acid)-R9; D-T-H-F-P-I-C-I-((S)-2-amino-4-phenylbutanoic acid)-R12; or D-T-H-F-P-I-C-I-((S)-2-amino-4-phenylbutanoic acid)-R13, wherein R8 is D-Pro-L-Pro-L-Lys; R9 is D-Pro-L-Pro-L-Arg; R10 is (L-β-homoproline)-L-Pro-L-Lys; R11 is (L-β-homoproline)-L-Pro-L-Arg; R12 is D-Pro-(Lβ-homoproline)-L-Lys; and R13 is D-Pro-(L-β-homoproline)-L-Arg.

A mini-hepcidin may comprise the amino acid sequence D-T-H-(3,3-diphenyl-L-alanine)-P-(D)R-C-(D)R-(3,3-diphenyl-L-alanine).

A mini-hepcidin may comprise the amino acid sequence C-(isonipecotic acid)-(3,3-diphenyl-D-alanine)-(4-(aminomethyl)cyclohexane carboxylic acid)-R-(4-(aminomethyl)cyclohexane carboxylic acid)-(isonipecotic acid)-(3,3-diphenyl-L-alanine)-cysteamide. A mini-hepcidin may comprise the amino acid sequence C-P-(3,3-diphenyl-D-alanine)-(4-(aminomethyl)cyclohexane carboxylic acid)-R-(4-(aminomethyl)cyclohexane carboxylic acid)-(isonipecotic acid)-(3,3-diphenyl-L-alanine)-cysteamide. A mini-hepcidin may comprise the amino acid sequence C-(D)P-(3,3-diphenyl-D-alanine)-(4-(aminomethyl)cyclohexane carboxylic acid)-R-(4-(aminomethyl)cyclohexane carboxylic acid)-(isonipecotic acid)-(3,3-diphenyl-L-alanine)-cysteamide. A mini-hepcidin may comprise the amino acid sequence C-G-(3,3-diphenyl-D-alanine)-(4-(aminomethyl)cyclohexane carboxylic acid)-R-(4-(aminomethyl)cyclohexane carboxylic acid)-(isonipecotic acid)-(3,3-diphenyl-L-alanine)-cysteamide.

In some embodiments, a mini-hepcidin has about 10% to 1000% of the activity of a 25 amino acid long peptide comprising the amino acid sequence set forth in SEQ ID NO: 1. For example, a mini-hepcidin may have about 50% to about 200% of the activity of a 25 amino acid long peptide comprising the amino acid sequence set forth in SEQ ID NO:1, such as about 75% to about 150% of the activity, about 80% to about 120% of the activity, about 90% to about 110% of the activity, or about 95% to about 105% of the activity. The term “activity” may refer to the ability of a mini-hepcidin to specifically bind to ferroportin, e.g., thereby inhibiting the transport of intracellular iron into the extracellular space, inhibiting the absorption of dietary iron, and/or reducing serum iron concentration. Activity may refer to the ability of a mini-hepcidin to inhibit the transport of intracellular iron into the extracellular space. Activity may refer to the ability of a mini-hepcidin to inhibit the absorption of dietary iron. Activity may refer to the ability of a mini-hepcidin to reduce serum iron concentration in vivo.

### Dosing

In certain aspects, provided herein are methods for treating or preventing iron overload in subject related to the administration to the subject hepcidin or a hepcidin analogue at an amount sufficient to reduce the serum iron concentration of the subject without inducing a serum iron level rebound. In certain aspects, provided herein are formulations for treating or preventing iron overload in subject comprising hepcidin or a hepcidin analogue in an amount sufficient to reduce the serum iron concentration of the subject without inducing a serum iron level rebound. In some embodiments, the amount of hepcidin or hepcidin analogue is such that the subject's transferrin saturation level is reduced to between 15% and 50% (e.g., between 20% and 50%, between 20% and 40%). In some embodiments, iron rebound is elevated serum iron levels post hepcidin or hepcidin analogue administration.

Provided herein are methods of treating or preventing iron overload in a subject and/or reducing serum iron levels in a subject by administering to the subject a hepcidin or hepcidin analogue in an amount sufficient to reduce the serum iron concentration of the subject. In some embodiments, administering the hepcidin or hepcidin analogue comprises administering the hepcidin or hepcidin analogue at an initial dose below the threshold dose to induce serum iron rebound. In some embodiments, the methods further comprise administering to the subject an additional dose of the hepcidin or hepcidin analogue. In some embodiments, the methods further comprise administering to the subject additional doses (e.g., at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50, 60, 70, 80, 90, 100, 150, or 200 additional doses) of the hepcidin or hepcidin analogue.

In some embodiments, the initial dose of the hepcidin or hepcidin analogue is from about 0.1 mg to about 40 mg, preferably from about 1 mg to about 30 mg (e.g., from about 5 mg to about 30 mg, from about 10 mg to about 30 mg, from about 20 mg to about 30 mg, from about 1 mg to about 20 mg, from about 5 mg to about 20 mg, from about 10 mg to about 20 mg, from about 1 mg to about 10 mg, from about 5 mg to about 10 mg). In some embodiments, the initial dose of the hepcidin or hepcidin analogue is about 0.1 mg, 0.5 mg, 1 mg, 2 mg, 2.5 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg or 40 mg.

In some embodiments, the additional dose or doses of the hepcidin or hepcidin analogue is from about 0.1 mg to about 40 mg, such as from about 1 mg to about 30 mg (e.g., from about 5 mg to about 30 mg, from about 10 mg to about 30 mg, from about 20 mg to about 30 mg, from about 1 mg to about 20 mg, from about 5 mg to about 20 mg, from about 10 mg to about 20 mg, from about 1 mg to about 10 mg, from about 5 mg to about 10 mg). In some embodiments, the additional dose or doses of the hepcidin or hepcidin analogue is about 0.1 mg, 0.5 mg, 1 mg, 2 mg, 2.5 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg or 40 mg.

The additional doses described herein may be the same, higher, or lower than the initial dose. Methods described herein may include increasing the dose of the hepcidin or hepcidin analogue between one dose and a subsequent dose. Methods described herein may include decreasing the dose of the hepcidin or hepcidin analogue between one dose and a subsequent dose. Methods described herein may include increasing the frequency of administering the hepcidin or hepcidin analogue (e.g., from biweekly to weekly, or weekly to semi-weekly). Methods described herein may include decreasing the frequency of administering the hepcidin or hepcidin analogue (e.g., from semi-weekly to weekly, or from weekly to biweekly). In some embodiments, the method further comprises measuring the patient's serum iron level. In some embodiments, the method comprises adjusting or titrating the dose of administration in response to a subject's serum iron measurement. In some embodiments, the methods comprise increasing the dose or frequency at which the hepcidin or hepcidin analogue is administered if serum iron levels are above a predetermined target level. In some embodiments, the methods comprise decreasing the dose or frequency at which the hepcidin or hepcidin analogue is administered if serum iron levels are below a predetermined target level. In some embodiments, the additional doses are the same as the initial doses. Comparing the assessment of serum iron after administering a dose to the assessment made prior to administering the dose will indicate whether serum iron is increasing or decreasing as a result of the hepcidin or hepcidin analogue therapy. In some embodiments, the amount of hepcidin or a hepcidin analogue administered to the subject or in the formulation is about 0.1 mg to about 40 mg. In some embodiments, the hepcidin or hepcidin analogue is administered in an amount of between about 1 mg to about 30 mg. In some embodiments, the amount is about 1000 μg, about 1100 μg, about 1200 μg about 1300 μg, about 1400 μg, about 1500 μg, about 1600 μg, about 1750 μg, about 1800 μg, about 2000 μg, about 2100 μg, about 2200 μg, about 2300 μg, about 2400 μg, about 2400 μg, about 2500 μg, about 2600 μg, about 2700 μg, about 2800 μg, about 2900 μg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg about 27 mg, about 28 mg, about 29 mg, or about 30 mg. In some embodiments, the amount of hepcidin or hepcidin analogue administered is at least about 1000 μg, about 1100 μg, about 1200 μg, about 1300 μg, about 1400 μg, about 1500 μg, about 1600 μg, about 1750 μg, about 1800 μg,about 2000 μg, about 2100 μg, about 2200 μg, about 2300 μg, about 2400 μg, about 2400 μg, about 2500 μg, about 2600 μg, about 2700 μg, about 2800 μg, about 2900 μg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg or about 20 mg. In some embodiments, the amount of hepcidin or hepcidin analogue administered is no more than about 20 mg, about 21 mg, about 22 mg, about 23 mg, about 24 mg, about 25 mg, about 26 mg about 27 mg, about 28 mg, about 29 mg, or about 30 mg.

In some embodiments, the subject is administered an individual dose (e.g., a bolus) of the amount of the hepcidin or hepcidin analogue. In some embodiments, the subject is administered multiple doses (e.g., 2, 3, 4, 5 or 6 doses) over a short period of time (e.g., 10 minutes, 20 minutes, 30 minutes, 40 minutes, 50 minutes, 1 hour, 2 hours, 3 hours, 4 hours, 5 hours, 6 hours, 7 hours, 8 hours, 9 hours, 10 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days) wherein the total amount of hepcidin or hepcidin analogue administered is in an amount sufficient to reduce the serum iron concentration of the subject without inducing a serum iron level rebound.

In some embodiments, the hepcidin or a hepcidin analogue is administered to the subject once per day, once every 2 days, once every 3 days, once every 4 days, once every 5 days, once every 6 days, once every 7 days, once every 8 days, once every 9 days, once every 10 days, once every 11 days, once every 12 days, once every 13 days or once every 14 days. In some embodiments, the hepcidin or a hepcidin analogue is administered to the subject 1, 2, 3, 4, 5, 6, or 7 times per week. In some embodiments, the hepcidin or a hepcidin analogue is administered to the subject 1, 2, or 3 times per week. In some embodiments, the hepcidin or a hepcidin analogue is administered to the subject once a week. In some embodiments, the hepcidin or a hepcidin analogue is administered to the subject once every two weeks.

In some embodiments, the amount of hepcidin or hepcidin analogue is administered as necessary. For example, in some embodiments, the amount of hepcidin or hepcidin analogue is administered when the subject's serum iron concentration elevates above a baseline serum iron concentration. In some embodiments, the hepcidin or hepcidin analogue is administered when the subject's transferrin saturation level rises above 50%, 55%, 60%, 65%, 70%, 75%, 80%, 85% or 90%.

In some embodiments, the methods comprise administering about 0.1 to about 40 mg of hepcidin or hepcidin analogue to the subject each time it is administered. In some embodiments, the methods comprise administering about 1 to about 30 mg of hepcidin or hepcidin analogue to the subject each time it is administered. In some embodiments, about 100 μg, about 200 μg, about 300 μg about 400 μg about 1500 μg, about 600 μg about 750 μg about 800 μg, about 1000 μg, about 1100 μg, about 1200μg, about 1300 μg, about 1400 μg, about 1500 μg, about 1600 μg, about 1750 μg, about 1800 μg, about 2000 μg, about 2100 μg, about 2200 μg, about 2300 μg, about 2400 μg, about 2400 μg, about 2500 μg, about 2600 μg, about 2700 μg, about 2800 μg, about 2900 μg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg about 27 mg, about 28 mg, about 29 mg, or about 30 mg of the hepcidin or hepcidin analogue is administered to the subject each time it is administered. In some embodiments, at least about 1000 μg, about 1100 μg, about 1200μg, about 1300 μg, about 1400 μg, about 1500 μg, about 1600 μg, about 1750 μg, about 1800 μg, about 2000 μg, about 2100 μg, about 2200 μg, about 2300 μg, about 2400 μg, about 2400 μg, about 2500 μg, about 2600 μg, about 2700 μg, about 2800 μg, about 2900 μg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg or about 20 mg of the hepcidin or hepcidin analogue is administered to the subject each time it is administered. In some embodiments, no more than about 20 mg, about 21 mg, about 22 mg, about 23 mg, about 24 mg, about 25 mg, about 26 mg about 27 mg, about 28 mg, about 29 mg, or about 30 mg of the hepcidin or hepcidin analogue is administered to the subject each time it is administered.

### Therapeutic Methods

In some aspects, provided herein are methods of treating and/or preventing iron overload in a subject, wherein the hepcidin or hepcidin analogue is administered at an amount sufficient to reduce the serum iron concentration of the subject without inducing a serum iron level rebound. In some embodiments, the method comprises administering to the subject pharmaceutical composition comprising hepcidin or a hepcidin analogue. In some embodiments, the subject has undergone iron chelation. In some embodiments, the subject has undergone phlebotomy therapy. In some embodiments, the subject is undergoing combination therapy with hepcidin or hepcidin analogue as well as iron chelation or phlebotomy therapy. The subject may have β-thalassemia, hemochromatosis, sickle cell disease, refractory anemia, or hemolytic anemia. The subject may be afflicted with hemochromatosis and the hemochromatosis may be hereditary hemochromatosis. The subject may have hemochromatosis and the hemochromatosis may be associated with hepatocarcinoma, cardiomyopathy, or diabetes. The subject may have anemia. Anemia may be, for example, a hemoglobinopathy, sideroblastic anemia, anemia associated with myelodysplastic syndrome (MDS), or a congenital anemia. The subject may have myelodysplastic syndrome (MDS). The subject may have hemoglobinopathy, sideroblastic anemia, or a congenital anemia. In some embodiments, the subject may have may be hepatocarcinoma, cardiomyopathy, or diabetes.

Investigators have found that iron chelators can improve outcomes in the treatment of infectious disease, such as malaria. See Cabantchik et al., Iron chelators as anti-infectives; malaria as a paradigm, FEMS Immunology and Medical Microbiology, 26 (1999) 289-298; Gordeuk et al., Effect of Iron Chelation Therapy on Recovery from Deep Coma in Children with Cerebral Malaria, N. Engl. J. Med. 1992, 327:1473-1477; Drakesmith, H. and Prentice, A., Viral infection and iron metabolism, Nat. Rev. Microbiol. 2008, 6:541-552; Iglesias-Osma et al., Iron metabolism and fungal infections in patients with haematological malignancies, J. Clin. Pathol. 1995, 48:223-225; B. J. Cherayil, The role of iron in the immune response to bacterial infection, Immunol. Res. 2011, 50:1-9. Accordingly, in some embodiments, the subject may have a viral, bacterial, fungal, or protist infection. In some embodiments, the subject may have a bacterial infection, and the bacteria is Escherichia coli, Mycobacterium (such as M. africanum, M. avium, M. tuberculosis, M. bovis, M. canetti, M. kansasii, M. leprae, M. lepromatosis, or M. microti), Neisseria cinerea, Neisseria gonorrhoeae, Staphylococcus epidermidis, Staphylococcus aureus, or Streptococcus agalactiae. In some embodiments, the subject may have a fungal infection, and the fungus is Candida albicans. In some embodiments, the subject may have a protist infection, and the protist is Trypanosoma cruzi, Plasmodium (such as P. falciparum, P. vivax, P. ovale, or P. malariae), Trypanosoma brucei (such as T. brucei gambiense or T. brucei rhodesiense), or Leishmania. The subject may have a viral, bacterial, fungal, or protist infection, and the viral, bacterial, fungal, or protist infection may be resistant to one or more agents for treating the viral, bacterial, fungal, or protist infection. The subject may have a bacterial infection and the bacterial infection may be tuberculosis. The subject may have Chagas disease, malaria, African sleeping sickness, or leishmaniasis. In some embodiments, the subject may have a viral infection, and the virus is hepatitis B, hepatitis C, or dengue virus.

In some embodiments, the method may comprise the conjoint administration of 4-aminosalicylic acid, aldesulfone, amikacin, amithiozone, bedaquiline, capreomycin, clofazimine, cycloserine, dapsone, delamanid, ethambutol, a fluoroquinolone, isoniazid, kanamycin, modified vaccinia Ankara 85A (MVA85A), morinamide, ofloxacin, pyrazinamide, recombinant Bacillus Calmette-Guérin 30 (rBCG30), rifampicin, rifater, streptomycin, terizidone, and/or thioacetazone to the subject. The method may comprise the conjoint administration of balofloxacin, cinoxacin, ciprofloxacin, clinafloxacin, danofloxacin, delafloxacin, difloxacin, enoxacin, enrofloxacin, fleroxacin, Fourth-generation, gatifloxacin, gemifloxacin, grepafloxacin, ibafloxacin, JNJ-Q2, levofloxacin, lomefloxacin, marbofloxacin, moxifloxacin, nadifloxacin, nalidixic acid, nemonoxacin, norfloxacin, ofloxacin, orbifloxacin, oxolinic acid, pazufloxacin, pefloxacin, pipemidic acid, piromidic acid, prulifloxacin, rosoxacin, rufloxacin, sarafloxacin, sitafloxacin, sparfloxacin, temafloxacin, tosufloxacin, and/or trovafloxacin to the subject. In certain such embodiments, the subject may have tuberculosis and/or a Mycobacterium infection. The subject may have tuberculosis and the tuberculosis may be drug-resistant tuberculosis.

In some embodiments, he subject may have tuberculosis and the tuberculosis may be multi-drug-resistant tuberculosis (MDR-TB), extensively drug-resistant tuberculosis (XDR-TB), or totally drug-resistant tuberculosis (TDR-TB). The subject may have tuberculosis, and the tuberculosis may not be drug-resistant, multi-drug-resistant, extensively drug-resistant, or totally drug-resistant. The subject may have tuberculosis and/or a Mycobacterium infection and subject may be resistant to isoniazid, ethambutol, rifampicin, pyrazinamide, ofloxacin, one or more fluoroquinolones, amikacin, kanamycin, and/or capreomycin.

In some embodiments, the method may comprise the conjoint administration of fluconazole, ketoconazole, miconazole, and/or itraconazole to the subject. In certain such embodiments, the subject has Chagas disease and/or Trypanosoma cruzi infection, and the subject has may be resistant to one or more of fluconazole, ketoconazole, miconazole, and/or itraconazole. The method may comprise the conjoint administration of fluconazole, benznidazole, and/or amphotericin B to the subject.

In some embodiments, the subject may have African sleeping sickness and the method may comprise conjointly administering an arsenical and/or diamidine to the subject. The subject may have African sleeping sickness and/or Trypanosoma bruce infection, and subject may be resistant to arsenicals and/or diamidines.

In some embodiments, the subject may have leishmaniasis and the methods herein may comprise conjointly administering a pentavalent antimonial to the subject. The subject may have leishmaniasis and the subject may be resistant to pentavalent antimonials. The methods may comprise conjointly administering amphotericin, amphotericin B, pentavalent antimonials, miltefosine, paromomycin, and/or fluconazole to the subject.

In some embodiments, the subject may have malaria. The subject may have malaria and the malaria may be resistant to one or more agents for treating malaria. The subject may have malaria, and the method may comprise conjoint administration of chloroquine, quinine, sulfadoxine-pyrimethamine, halofantrine, atovaquone, and/or mefloquine to the subject. The subject may have malaria, and the malaria may be resistant to one or more of chloroquine, quinine, sulfadoxine-pyrimethamine, halofantrine, atovaquone, and/or mefloquine. The subject may have a multidrug-resistant falciparum malaria infection. The method may comprise the conjoint administration of one or more of proguanil, chlorproguanil, pyronaridine, lumefantrinel, mefloquine, dapsone, atovaquone, and/or artesunate to the subject.

In some embodiments, the method may comprise the conjoint administration of artemisinin or an artemisinin derivative to the subject. The method may comprise the conjoint administration of artesunate, artemisinin, dihydro-artemisinin, artelinate, arteether, and/or artemether to the subject.

In some embodiments, the hepcidin or hepcidin analogues can be administered in a variety of conventional ways. In some aspects, the hepcidin or hepcidin analogues are suitable for parenteral administration. These hepcidin or hepcidin analogues may be administered, for example, intraperitoneally, intravenously, intrarenally, or intrathecally. In some aspects, the hepcidin or hepcidin analogues are injected intravenously.

In some embodiments, the hepcidin or hepcidin analogues may be administered topically, enterally, or parenterally. Hepcidin or hepcidin analogues may be administered subcutaneously, intravenously, intramuscularly, intranasally, by inhalation, orally, sublingually, by buccal administration, topically, transdermally, or transmucosally. Hepcidin or hepcidin analogues may be administered by injection. In some embodiments, hepcidin or hepcidin analogues is administered by subcutaneous injection, orally, intranasally, by inhalation, or intravenously. In certain embodiments, the hepcidin or hepcidin analogues is administered by subcutaneous injection.

In some embodiments, the subject may be a mammal. In some embodiments, the subject may be a rodent, lagomorph, feline, canine, porcine, ovine, bovine, equine, or primate. In certain embodiments, the subject is a human. In some embodiments, the subject may be a female or male. In some embodiments, the subject may be an infant, child, or adult.

In some embodiments, the serum iron concentration of the subject is at least about 50 μg/dL prior to administering the hepcidin or hepcidin analogue, such as at least about 55 μg/dL, at least about 60 μg/dL, at least about 65 μg/dL, at least about 70 μg/dL, at least about 75 μg/dL, at least about 80 μg/dL, at least about 85 μg/dL, at least about 90 μg/dL, at least about 95 μg/dL, at least about 100 μg/dL, at least about 110 μg/dL, at least about 120 μg/dL, at least about 130 μg/dL, at least about 140 μg/dL, at least about 150 μg/dL, at least about 160 μg/dL, at least about 170 μg/dL, at least about 175 μg/dL, at least about 176 μg/dL, at least about 177 μg/dL, at least about 180 μg/dL, at least about 190 μg/dL, at least about 200 μg/dL, at least about 210 μg/dL, at least about 220 μg/dL, at least about 230 μg/dL, at least about 240 μg/dL, at least about 250 μg/dL, at least about 260 μg/dL, at least about 270 μg/dL, at least about 280 μg/dL, at least about 290 μg/dL, or at least about 300 μg/dL. The serum iron concentration of the subject may be about 50 μg/dL to about 500 μg/dL prior to administering the hepcidin or hepcidin analogue, such as about 55 μg/dL to about 500 μg/dL, about 60 μg/dL to about 500 μg/dL, about 65 μg/dL to about 500 μg/dL, about 70 μg/dL to about 500 μg/dL, about 75 μg/dL to about 500 μg/dL, about 80 μg/dL to about 500 μg/dL, about 85 μg/dL to about 500 μg/dL, about 90 μg/dL to about 500 μg/dL, about 95 μg/dL to about 500 μg/dL, about 100 μg/dL to about 500 μg/dL, about 110 μg/dL to about 500 μg/dL, about 120 μg/dL to about 500 μg/dL, about 130 μg/dL to about 500 μg/dL, about 140 μg/dL to about 500 μg/dL, about 150 μg/dL to about 500 μg/dL, about 160 μg/dL to about 500 μg/dL, about 170 μg/dL to about 500 μg/dL, about 175 μg/dL to about 500 μg/dL, about 176 μg/dL to about 500 μg/dL, about 177 μg/dL to about 500 μg/dL, about 180 μtg/dL to about 500 μg/dL, about 190 μg/dL to about 500 μg/dL, about 200 μg/dL to about 500 μg/dL, about 210 μg/dL to about 500 μg/dL, about 220 μg/dL to about 500 μg/dL, about 230 μg/dL to about 500 μg/dL, about 240 μg/dL to about 500 μg/dL, about 250 μg/dL to about 500 μg/dL, about 260 μg/dL to about 500 μg/dL, about 270 μg/dL to about 500 μg/dL, about 280 μg/dL to about 500 μg/dL, about 290 μg/dL to about 500 μg/dL, or about 300 μg/dL to about 500 μg/dL.

In some embodiments, administering the hepcidin or hepcidin analogue to a subject decreases the serum iron concentration of the subject. In some embodiments, the serum iron concentration of the subject is less than about 200 μg/dL following administration the hepcidin or hepcidin analogue, such as less than about 200 μg/dL, less than about 195 μg/dL, less than about 190 μg/dL, less than about 185 μg/dL, less than about 180 μg/dL, less than about 175 μg/dL, less than about 170 μg/dL, such as less than about 165 μg/dL, less than about 160 μg/dL, less than about 155 μg/dL, less than about 150 μg/dL, less than about 145 μg/dL, less than about 140 μg/dL, less than about 135 μg/dL, such as less than about 130 μg/dL, less than about 125 μg/dL, less than about 120 μg/dL, less than about 115 μg/dL, less than about 110 μg/dL, or less than about 105 μg/dL. The serum iron concentration of the subject is less than about 100 μg/dL following administration the hepcidin or hepcidin analogue, such as less than such as less than about 95 μg/dL, less than about 90 μg/dL, less than about 85 μg/dL, less than about 80 μg/dL, less than about 75 μg/dL, less than about 70 μg/dL, such as less than about 65 μg/dL, less than about 60 μg/dL, less than about 55 μg/dL, less than about 50 μg/dL, less than about 45 μg/dL, less than about 40 μg/dL, less than about 35 μg/dL, such as less than about 30 μg/dL, less than about 25 μg/dL, less than about 20 μg/dL, less than about 15 μg/dL, less than about 10 μg/dL, or less than about 5 μg/dL.

In some embodiments, administering the hepcidin or hepcidin analogue may decrease the serum iron concentration of the subject for at least 24 hours, at least 2 days, at least 3 days, at least 4 days, at least 5 days, at least 6 days or at least 7 days. For example, administering the hepcidin or hepcidin analogue may decrease the serum iron concentration of the subject by at least about 5 μg/dL. Administering the hepcidin or hepcidin analogue may decrease the serum iron concentration of the subject by at least about 5 μg/dL for at least 4 hours, at least 6 hours, at least 8 hours, at least 12 hours, at least 24 hours, or at least 48 hours. Administering the hepcidin or hepcidin analogue may decrease the serum iron concentration of the subject by at least about 5 μg/dL for at least 1 day, at least 2 days, at least 3 days, at least 4 days, at least 5 days, at least 6 days, or at least 7 days. Administering the hepcidin or hepcidin analogue may decrease the serum iron concentration of the subject by at least about 5%, such as at least about 10%, at least about 15%, at least about 20%, at least about 25%, at least about 30%, at least about 35%, at least about 40%, at least about 45%, at least about 50% or at least about 55%. Administering the hepcidin or hepcidin analogue may decrease the serum iron concentration of the subject by at least about 5% for at least 4 hours, at least 6 hours, or at least 12 hours. Administering the hepcidin or hepcidin analogue may decrease the serum iron concentration of the subject by at least about 5%, 10% or 15% for at least 1 day, at least 2 days, at least 3 days, at least 4 days, at least 5 days, at least 6 days, or at least 7 days.

In some embodiments, the subject has a serum hepcidin concentration of less than about 1000 ng/mL prior to administering the hepcidin or hepcidin analogue, such as less than about 900 ng/mL, less than about 800 ng/mL, less than about 700 ng/mL, less than about 600 ng/mL, less than about 500 ng/mL, less than about 400 ng/mL, less than about 300 ng/mL, less than about 200 ng/mL, less than about 100 ng/mL, less than about 90 ng/mL, less than about 80 ng/mL, less than about 70 ng/mL, less than about 60 ng/mL, less than about 50 ng/mL, less than about 40 ng/mL, less than about 30 ng/mL, less than about 20 ng/mL, or less than about 10 ng/mL. The subject may have a serum hepcidin concentration of about 1 ng/mL to about 1000 ng/mL prior to administering the hepcidin or hepcidin analogue, such as about 1 ng/mL to about 900 ng/mL, about 1 ng/mL to about 800 ng/mL, about 1 ng/mL to about 700 ng/mL, about 1 ng/mL to about 600 ng/mL, about 1 ng/mL to about 500 ng/mL, about 1 ng/mL to about 400 ng/mL, about 1 ng/mL to about 300 ng/mL, about 1 ng/mL to about 200 ng/mL, about 1 ng/mL to about 100 ng/mL, about 1 ng/mL to about 90 ng/mL, about 1 ng/mL to about 80 ng/mL, about 1 ng/mL to about 70 ng/mL, about 1 ng/mL to about 60 ng/mL, about 1 ng/mL to about 50 ng/mL, about 1 ng/mL to about 40 ng/mL, about 1 ng/mL to about 30 ng/mL, about 1 ng/mL to about 20 ng/mL, or about 1 ng/mL to about 10 ng/mL.

In some embodiments, the subject has a serum ferritin concentration greater than about 10 ng/mL prior to administering the hepcidin or hepcidin analogue, such as greater than about 20 ng/mL, greater than about 30 ng/mL, greater than about 40 ng/mL, greater than about 50 ng/mL, greater than about 60 ng/mL, greater than about 70 ng/mL, greater than about 80 ng/mL, greater than about 90 ng/mL, greater than about 100 ng/mL, greater than about 200 ng/mL, greater than about 300 ng/mL, greater than about 400 ng/mL, greater than about 500 ng/mL, greater than about 600 ng/mL, greater than about 700 ng/mL, greater than about 800 ng/mL, greater than about 900 ng/mL, greater than about 1000 ng/mL, greater than about 2000 ng/mL, greater than about 3000 ng/mL, greater than about 4000 ng/mL, greater than about 5000 ng/mL, greater than about 6000 ng/mL, greater than about 7000 ng/mL, greater than about 8000 ng/mL, greater than about 9000 ng/mL, or even greater than about 10 μg/mL. The subject may have a serum ferritin concentration of about 10 ng/mL to about 100 μg/mL prior to administering hepcidin or hepcidin analogue, such as about 20 ng/mL to about 100 μg/mL, about 30 ng/mL to about 100 μg/mL, about 40 ng/mL to about 100 μg/mL, about 50 ng/mL to about 100 μg/mL, about 60 ng/mL to about 100 μg/mL, about 70 ng/mL to about 100 μg/mL, about 80 ng/mL to about 100 μg/mL, about 90 ng/mL to about 100 μg/mL, about 100 ng/mL to about 100 μg/mL, about 200 ng/mL to about 100 μg/mL, about 300 ng/mL to about 100 μg/mL, about 400 ng/mL to about 100 μg/mL, about 500 ng/mL to about 100 μg/mL, about 600 ng/mL to about 100 μg/mL, about 700 ng/mL to about 100 μg/mL, about 800 ng/mL to about 100 μg/mL, about 900 ng/mL to about 100 μg/mL, or about 1000 ng/mL to about 100 μg/mL. The subject may have a serum ferritin concentration of about 10 ng/mL to about 20 μg/mL prior to administering hepcidin or hepcidin analogue, such as about 20 ng/mL to about 20 μg/mL, about 30 ng/mL to about 20 μg/mL, about 40 ng/mL to about 20 μg/mL, about 50 ng/mL to about 20 μg/mL, about 60 ng/mL to about 20 μg/mL, about 70 ng/mL to about 20 μg/mL, about 80 ng/mL to about 20 μg/mL, about 90 ng/mL to about 20 μg/mL, about 100 ng/mL to about 20 μg/mL, about 200 ng/mL to about 20 μg/mL, about 300 ng/mL to about 20 μg/mL, about 400 ng/mL to about 20 μg/mL, about 500 ng/mL to about 20 μg/mL, about 600 ng/mL to about 20 μg/mL, about 700 ng/mL to about 20 μg/mL, about 800 ng/mL to about 20 μg/mL, about 900 ng/mL to about 20 μg/mL, or about 1000 ng/mL to about 20 μg/mL.

In some embodiments, the subject has a serum ferritin concentration of less than about 10 μg/mL prior to administering hepcidin or hepcidin analogue, such as less than about 1000 ng/mL, less than about 900 ng/mL, less than about 800 ng/mL, less than about 700 ng/mL, less than about 600 ng/mL, less than about 500 ng/mL, less than about 400 ng/mL, less than about 300 ng/mL, less than about 200 ng/mL, less than about 100 ng/mL, less than about 90 ng/mL, less than about 80 ng/mL, less than about 70 ng/mL, less than about 60 ng/mL, less than about 50 ng/mL, less than about 40 ng/mL, less than about 30 ng/mL, less than about 20 ng/mL, or less than about 10 ng/mL. The subject may have a serum ferritin concentration of about 1 ng/mL to about 1000 ng/mL prior to administering the hepcidin or hepcidin analogue, such as about 1 ng/mL to about 900 ng/mL, about 1 ng/mL to about 800 ng/mL, about 1 ng/mL to about 700 ng/mL, about 1 ng/mL to about 600 ng/mL, about 1 ng/mL to about 500 ng/mL, about 1 ng/mL to about 400 ng/mL, about 1 ng/mL to about 300 ng/mL, about 1 ng/mL to about 200 ng/mL, about 1 ng/mL to about 100 ng/mL, about 1 ng/mL to about 90 ng/mL, about 1 ng/mL to about 80 ng/mL, about 1 ng/mL to about 70 ng/mL, about 1 ng/mL to about 60 ng/mL, about 1 ng/mL to about 50 ng/mL, about 1 ng/mL to about 40 ng/mL, about 1 ng/mL to about 30 ng/mL, about 1 ng/mL to about 20 ng/mL, or about 1 ng/mL to about 10 ng/mL.

In some embodiments, administering the hepcidin or hepcidin analogue decreases the serum ferritin concentration of the subject. For example, administering hepcidin or hepcidin analogue may decrease the serum ferritin concentration of the subject to about less than 100 ng/mL after to administering the hepcidin or hepcidin analogue to the subject, such as about less than 90 ng/mL, about less than 85 ng/mL, about less than 80 ng/mL, about less than 75 ng/mL, about less than 70 ng/mL, about less than 65 ng/mL, about less than 60 ng/mL, or about less than 55 ng/mL. In some embodiments, administering hepcidin or hepcidin analogue may decrease the serum ferritin concentration of the subject to about less than 50 ng/mL after to administering the hepcidin or hepcidin analogue to the subject, such as about less than 45 ng/mL, about less than 40 ng/mL, about less than 35 ng/mL, about less than 30 ng/mL, about less than 25 ng/mL, about less than 20 ng/mL, or about less than 15 ng/mL. Administering hepcidin or hepcidin analogue may decrease the serum ferritin concentration of the subject by at least 10 ng/mL, at least about 20 ng/mL, at least about 30 ng/mL, at least about 40 ng/mL, at least about 50 ng/mL, at least about 60 ng/mL, at least about 70 ng/mL, at least about 80 ng/mL, at least about 90 ng/mL, or at least about 100 ng/mL.

In some embodiments, the subject has a total body iron content of about 40 to about 50 mg/kg prior to administering the hepcidin or hepcidin analogue. The subject may have a total body iron content greater than about 50 mg/kg prior to administering hepcidin or hepcidin analogue, such as greater than about 55 mg/kg, greater than about 60 mg/kg, greater than about 65 mg/kg, or greater than about 70 mg/kg. In some embodiments, the subject has a total body iron content of less than 25 mg/kg after to administering the hepcidin or hepcidin analogue to the subject, such as less than 24 mg/kg, such as less than 23 mg/kg, such as less than 22 mg/kg, such as less than 21 mg/kg, such as less than 20 mg/kg, such as less than 19 mg/kg, such as less than 18 mg/kg, such as less than 17 mg/kg, such as less than 16 mg/kg, such as less than 15 mg/kg, such as less than 14 mg/kg, such as less than 13 mg/kg, such as less than 12 mg/kg, such as less than 11 mg/kg, such as less than 10 mg/kg, such as less than 9 mg/kg, such as less than 8 mg/kg, such as less than 7 mg/kg, such as less than 6 mg/kg, or such as less than 5 mg/kg.

In some embodiments, the subject has a transferrin saturation percentage greater than about 50% prior to administering hepcidin or hepcidin analogue, such as greater than about 55%, greater than about 60%, greater than about 65%, greater than about 70%, greater than about 75%, greater than about 80%, greater than about 85%, greater than about 90%, greater than about 95%. The subject may have a transferrin saturation percentage of about 50% to about 99% prior to administering hepcidin or hepcidin analogue, such as about 55% to about 99%, about 60% to about 99%, about 65% to about 99%, about 70% to about 99%, about 75% to about 99%, about 80% to about 99%, about 85% to about 99%, about 90% to about 99% or about 95% to about 99%.

In some embodiments, administering the hepcidin or hepcidin analogue decreases the transferrin saturation percentage of the subject. For example, administering hepcidin or hepcidin analogue to a subject may decrease the transferrin saturation percentage of the subject to between 15% and 50%, to between 15% and 25%, to between 20% and 50%, to between 20% and 40%, to between 25% and 50%, to between 25% and 40%, to between 30% and 50% or to between 30% and 40%.

In certain particular embodiments, the method comprises subcutaneously administering an initial 5-mg dose of hepcidin followed by subsequent weekly doses. The subsequent doses can be increased or decreased to determine the optimal dose, based on the patient's clinical response and/or predetermined target parameters. For example, the weekly dose can be increased up to about 20 mg (e.g., by raising the dose per administration up to about 10 mg and increasing the dosing frequency up to twice weekly) or even up to about 40 mg or reduced down to about 1 mg (e.g., by lowering the dose per administration and/or decreasing the dosing frequency down to biweekly). For example, the dose of hepcidin can be titrated to achieve a TSAT level from 20% to 50%. In certain preferred embodiments, the subject has hemochromatosis, such as hereditary hemochromatosis, or beta-thalassemia.

### EXEMPLIFICATION

### EXAMPLE 1

### Serum Iron Rebound Following Administration of a Threshold Level of Hepcidin

Patients were recruited for a multi-day study to observe serum iron levels following administration of hepcidin. All patients who were recruited had either refractory or hemolytic anemia or hemochromatosis. Patients are divided into cohorts and subsequently treated with hepcidin. Treatment consisted of a single subcutaneous (SC) dose of hepcidin on Day 1. Dose escalation with new cohorts occurred only after the patients at each dose level had been observed for a minimum of 3 days and no drug related toxicity had been observed. The starting dose level was 1 mg given via SC injection on Day 1. Doses ranged from 1 mg to 30 mg. Patient cohorts and dosing can be found in Table 1:

TABLE 1
Dose Escalation:

| | | |
|---|---|---|
| Dose Level | Dose (Day 1) | No. Patients |

| | | | |
|---|---|---|---|
| 1 | 1 | mg | 3 |
| 2 | 5 | mg | 3 |
| 3 | 10 | mg | 3 |
| 4 | 20 | mg | 6 |
| 5 | 30 | mg | 3 |

Patients were admitted to the research unit the day before the administration of study drug and discharged following assessments 48 hours post dose. Serum iron, ferritin, transferrin, total iron binding capacity, and unsaturated iron binding capacity were measured on Day 1(baseline) and Day 7 (end of study). Serum iron was also measured at 2, 4, 8, 24, and 48 hours post dosing. The percent change in Serum Iron from baseline to hour 8 is provided in Table 2.

TABLE 2
Change in serum iron at 8 hours

| | |
|---|---|
| | Percent Change in Serum Iron |
| Dose Group | from Baseline to Hour 8 |

| | | |
|---|---|---|
| 1 | mg (n = 3) | −14.2% (p = 0.203) |
| 5 | mg (n = 3) | −26.7% (p = 0.380) |
| 10 | mg (n = 3) | −45.5% (p = 0.077) |
| 20 | mg (n = 6) | −55.7% (p = 0.001) |

P-values not adjusted for potential regression to the mean effect

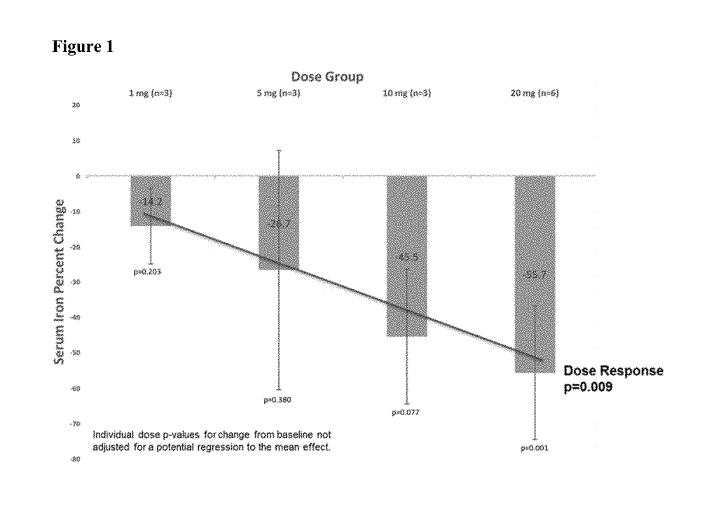

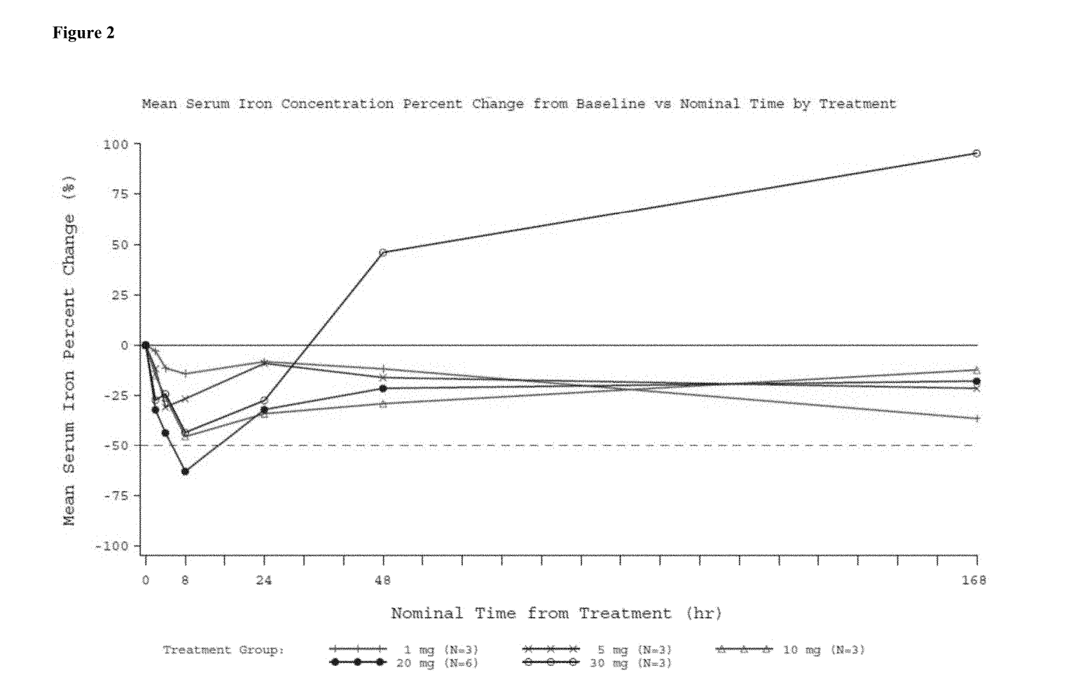

As seen in FIG. 1, a dose-dependent, statistically significant reduction in serum iron was observed (p-0.009 for dose response). At the 20 mg dose level, hepcidin reduced serum iron by 55.7% from baseline to hour 8 (p=0.001; not adjusted for potential regression to the mean effect). As seen in FIG. 2, for subjects administered 1 mg, 5 mg, 10 mg or 20 mg of hepcidin, serum iron had not returned to baseline through day 7 (18.0% reduction from baseline to the end of day 7). Counterintuitively, subjects administered at least 30 mg of hepcidin experienced a rebound effect which resulted in above-baseline serum iron levels within 48 hours of treatment.

### EXAMPLE 2

### Hepcidin Administration in Patients at Risk for Iron Overload Disorders

Eighteen patients at risk for iron overload disorders (i.e., refractory or hemolytic anemia or hemochromatosis) received one or two 1-mL subcutaneous injections to achieve the desired dose level (1 mg, 5 mg, 10 mg, 20 mg, or 30 mg). Hepcidin concentration ranged from 1 to 15 mg/mL.

A decrease in serum iron levels compared to baseline was generally observed in all five dose groups. Mean maximum percentage reductions per dose group occurred at 8 hours post-dose and reached 58% in the 20-mg dose group. A significant dose response was present, with greater decrease in serum iron at higher doses. The serum iron reduction was sustained up to Day 8 in most patients. Decreases in ferritin, total iron binding capacity (TIBC), transferrin, and TSAT % levels were also observed from Baseline (pre-dose) to Day 8/End of Study (EOS). No anti-drug antibodies were detected.

### EXAMPLE 3

### Hepcidin Administration in Healthy Subjects

This study enrolled 32 healthy adult subjects in 4 dose groups of 8 subjects per cohort, with 6 subjects assigned to hepcidin treatment and 2 subjects assigned to placebo in each cohort. The starting dose was 5 mg of hepcidin or placebo; subsequent dose groups received 10 mg, 20 mg, and 30 mg, respectively. Subjects received between one and three 1-mL subcutaneous (SC) injections to deliver the desired dose level. Hepcidin concentration was either 5 mg/mL or 10 mg/mL. For data analysis purposes, placebo subjects for all cohort groups were pooled.

A decrease in serum iron levels compared to baseline was observed in all four dose groups, with mean maximum reduction of 33% to 65% at 8-hours post dose. Serum iron levels returned to baseline before 48 hours. A significant dose response was observed, with larger reductions in serum iron generally associated with increases in dose up to 20 mg. There was no apparent difference in the maximum reduction between the 20 and 30 mg dose levels. Ferritin, total iron binding capacity (TIBC), transferrin, and TSAT % were increased from baseline at Day 8 for all treatment groups. Unsaturated iron binding capacity (UIBC) levels showed no trends in changes from baseline at Day 8. No anti-drug antibodies were detected.

### EXAMPLE 4

### Hepcidin Administration in Patients with Hereditary Hemochromatosis

One hundred and twenty patients with hereditary hemochromatosis are administered hepcidin. Patients are randomized 2:1 to Hepcidin or Placebo. Patients are administered hepcidin or placebo subcutaneously for 12 weeks. All patients receive standard of care therapeutic phlebotomy within 10 to 14 days after the first dose of study drug.

Initial dose of hepcidin is 5 mg. Patient dose is increased or decreased to determine the optimal dose, considering efficacy and safety parameters. The maximum weekly dose is 20 mg while the minimum weekly dose is 1 mg. In addition, the dose amount of hepcidin is titrated in order to achieve the desired TSAT level (20% to 50%) or maximally tolerated dose. TSAT levels are measured weekly during the treatment period. Patients are dosed once weekly dependent on the patient's previous TSAT reading in relation to the next planned dose.

### EXAMPLE 5

### Hepcidin Administration in Patients with Beta-Thalassemia

One hundred patients receive hepcidin either as immediate treatment or as deferred treatment. The deferred treatment group includes an on-study observation period of standard of care (SOC) therapy being administered at study entry.

Eligible patients are randomized in a 1:1 ratio to treatment group A or B:

Group A (Deferred Therapy)
A1: 26 weeks SOC
A2: 26 weeks hepcidin and SOC

Group B (Immediate Therapy)
B: 52 weeks hepcidin and SOC

Patients in Group A receive SOC and observation for 26 weeks followed by hepcidin plus SOC and observation for 26 weeks. Patients in Group B receive hepcidin plus SOC and observation for 52 weeks. Patients are dosed with hepcidin (administered SC) for 26 weeks (Group A) or 52 weeks (Group B).

The initial dose of hepcidin is 5 mg. The maximum weekly dose is 40 mg while the minimum weekly dose is 1 mg. A patient may have their dose increased or decreased to determine the proper dose, considering efficacy and safety parameters. The dose amount of hepcidin is titrated in order to achieve the desired TSAT level (<50% and >20%) or maximally tolerated dose. During the treatment period, TSAT levels will be measured at each of the first 4 weeks and at 3- to 4-week intervals thereafter. Patients are dosed once weekly dependent on the patient's most recent TSAT % reading in relation to the next planned dose; but for patients who require more or less frequent dosing, the schedule can be as often as twice weekly or as few as every other week.

1-88. (canceled)

89. A method of treating or preventing iron overload in a subject and/or reducing serum iron level in a subject, comprising administering a hepcidin or hepcidin analogue at an initial dose at or below 40 mg that is sufficient to reduce the serum iron concentration of the subject.

90. The method of claim 89, wherein the initial dose is from about 0.1 mg to about 40 mg of the hepcidin or hepcidin analogue.

91-101. (canceled)

102. The method of claim 89, wherein the initial dose is sufficient to reduce the transferrin saturation level in the subject to between 20% and 50%.

103-107. (canceled)

108. The method of claim, further comprising administering to the subject one or more additional doses of the hepcidin or hepcidin analogue.

109. The method of claim 89, wherein administering the hepcidin or hepcidin analogue comprises administering additional doses of the hepcidin or hepcidin analogue at a frequency of about twice a week, once a week, once every 10 days or once every 2 weeks.

110. The method of claim 109, wherein administering the hepcidin or hepcidin analogue comprises administering the hepcidin or hepcidin analogue about once a week.

111. (canceled)

112. The method of claim 109, wherein each additional dose is from about 0.1 mg to about 40 mg of hepcidin.

113-115. (canceled)

116. The method of claim 112, wherein each additional dose is about 2.5 mg, about 5 mg, about 10 mg, about 15 mg, about 20 mg, about 25 mg, about 30 mg, about 35 mg, or about 40 mg of hepcidin or hepcidin analogue.

117-125. (canceled)

126. The method of claim 112, wherein each additional dose is the same as the initial dose.

127. The method of claim 112, wherein each additional dose is higher than the initial dose.

128. The method of claim 112, further comprising increasing the frequency of administering the additional doses.

129. The method of 112, further comprising decreasing the frequency of administering the additional doses.

130-133. (canceled)

134. The method of claim 89, wherein the hepcidin or hepcidin analogue is administered by injection.

135. The method of claim 134, wherein the hepcidin or hepcidin analogue is administered by subcutaneous injection.

136. The method of claim 135, wherein the subject has β-thalassemia, hemochromatosis, or anemia, such as refractory anemia, or hemolytic anemia.

137. The method of claim 136, wherein the subject has β-thalassemia.

138. (canceled)

139. The method of claim 136, wherein the subject has hereditary hemochromatosis.

140-172. (canceled)

173. The method of claim 89, wherein the transferrin saturation of the subject is decreased to between 20% and 50% following administration the hepcidin or hepcidin analogue to the subject.

174. (canceled)

175. The method of claim 89, wherein the hepcidin or hepcidin analogue has at least 80% sequence homology to SEQ ID NO 1.

176. The method of claim 89, wherein the hepcidin or hepcidin analogue has 100% sequence homology to SEQ ID NO 1.

177-198. (canceled)
